(19) United States
(12) Patent Application Publication
(10) Pub. No.: US 2003/0099622 A1
(43) Pub. Date: May 29, 2003

(54) Systems and methods for inducing mixed chimerism

(75) Inventors: Bernhard Hering, Minnetonka, MN (US); Bruce R. Blazar, Golden Valley, MN (US); James V. Harmon, Minneapolis, MN (US); Nicole Kirchhof, Minnetonka, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: The Regents of the University of Minnesota00000000

(21) Appl. No.: 10/124,617

(22) Filed: Apr. 16, 2002

Related U.S. Application Data
(63) Continuation-in-part of application No. 09/855,027, filed on May 14, 2001.
(60) Provisional application No. 60/284,005, filed on Apr. 16, 2001.

(51) Int. Cl. A61K 48/00; C12N 5/08

(52) U.S. Cl. 424/93.21; 435/366

(57) ABSTRACT

A mixed chimeric immune system is created for a variety of treatments and techniques. Mixed chimerism is established in a recipient without significant risk of profound neutropenia or graft-versus-host-disease (GVHD) by administering a cell transplant from a donor to a recipient along with a conditioning treatment and an immune blockade treatment.

### RELATED APPLICATIONS

This application is a continuation-in-part of U.S. patent application Ser. No. 09/855,207 filed Jan. 29, 2002, “Systems And Methods For Inducing Mixed Chimerism” which claims priority to U.S. provisional patent application Serial No. 60/284,005 filed Apr. 16, 2001, which priority documents are hereby incorporated herein by reference.

### FIELD OF THE INVENTION

The invention relates to inducing tolerance to transplanted materials such as allogeneic, xenogeneic, and autogeneic materials transplanted into a patient and to restoring self-tolerance in the case of autoimmunity conditions. More specifically, the invention relates to creating mixed chimerism in patients and treating graft rejection, malignant cell growth, and autoimmune conditions.

### BACKGROUND OF THE INVENTION

Organ transplantation has saved many lives and greatly improved the quality of life for organ recipients; however, the recipients must be treated for the rest of their lives with powerful drugs that suppress their immune system. Unfortunately, these immunosuppressant drugs make the recipient vulnerable to disease and block the body's natural cancer resistance. While the immunosuppressant drugs are designed to prevent rejection of the transplanted organ, these drugs are not always effective and transplanted organs are often rejected after a short time (acute rejection) or over the long term (chronic rejection). For instance, only about 50% of heart, lung, or liver transplants that function after one year are still functioning at ten years.

The ability for a patient to successfully tolerate transplanted organs is referred to as tolerance. Just as the human body's immune system normally tolerates its own organs, a condition called self-tolerance, an organ recipient would ideally tolerate a donated organ without the need for long-term immunosuppressant drugs. Tolerance without the need for continued use of such immunosuppressant drugs is one of the principle goals of the field of transplantation. While many attempts are being made to achieve this goal, the understanding of the immune system is still incomplete and no approach has yet to reach this goal in a manner suitable for a clinical setting.

T-cells are the immune system cells that are chiefly responsible for transplant rejection and autoimmune disorders. One approach to achieving tolerance has been to destroy a recipient's bone marrow cells, which produce the T-cells, and completely replace them with a donor's bone marrow. The destruction of bone marrow is termed myeloablation. Since bone marrow plays a key role in the immune system, the recipient begins to use the “donated” immune system. The complete myeloablation and replacement of bone marrow causes the recipient to use only the donated immune system, a condition termed full chimerism. The major obstacle to successful bone marrow transplantation is the toxicity associated with myeloablation and graft-versus-host disease (GVHD). Myeloablation weakens the immune system and makes a patient vulnerable to infections. GVHD is a common complication of allogeneic bone marrow transplants (i.e., bone marrow transplants from a donor other than an identical twin). GVHD is a condition where the donor's bone marrow, especially its T-cells, attack the patient's own organs and tissue, including the skin, liver, and gastrointestinal tract. A severe case of GVHD is often fatal.

Another approach to creating tolerance has been to use agents to directly block the T-cell response to the transplanted organ. The T-cell response includes the interaction of molecules on the surface of the T-cells with molecules on other cells. The T-cells have certain molecules, (e.g., CD 154 and CD28) that interact with receptor molecules in other cells (e.g., the CD40 receptor and the B7 receptor molecules, respectively). Drugs that block these interactions (anti-CD154 antibody, which blocks the CD154-to-CD40 receptor interaction and CTLA4Ig, which interferes with CD28-to-B7 interaction) can interfere with the organ rejection process. While high levels of anti-CD154 antibody have been reported to block GVHD, the level of these drugs necessary to completely interfere with the organ rejection process can create problems similar to conventional immunosuppressant drugs.

Recently, it has been suggested that tolerance might be achieved as a result of successfully inducing a condition termed mixed chimerism. In mixed chimerism, the recipient would use both their original immune system and a donated immune system. The donor and recipient immune systems would co-exist and cooperate in the recipient. In addition to potentially creating tolerance for transplants, the ability to successfully establish mixed chimerism could be used as a therapy for autoimmune diseases. Part of the challenge of creating mixed chimerism, however, is that the donor and recipient T-cells initiate immune systems attack each other or the recipient, which can result in GVHD. Although mixed chimerism should reduce the risks of GVHD compared to full chimerism, scientists have yet to discover how to consistently and safely establish mixed chimerism without generating GVHD.

Several approaches for establishing mixed chimerism have been attempted. In general, these approaches use techniques that severely suppress the functions of the recipient's bone marrow and/or immune system for a prolonged period of time as part of the treatment. Such severe and lengthy suppression has been thought necessary to let donor and recipient T-cells adapt to a state of coexistence. Suppression of bone marrow and immune functions is typically achieved with irradiation therapy and/or high doses of drugs such as fludarabine phosphate, cyclophosphamide, and busulfan. An important measure of severe suppression is whether the patient exhibits neutropenia, a condition indicating a shortage of neutrophils (white blood cells that digest and destroy particles and fight infections).

Suppression of the immune system, however, is undesirable because it leaves patients vulnerable to opportunistic infections and disease during the course of such treatments. As a result, the rate of complications and the cost of treatment are increased. Suppression of the bone marrow not only suppresses the immune system but also suppresses the body's ability to make blood (termed hematopoiesis). Damage to the blood-making ability severely impacts the recipient's health.

Removal of T-cells from donor marrow is another typical step that has been attempted in an effort to help prevent GVHD. The concept behind this step is that removing most of the donor T-cells will decrease the risk of an attack on the recipient by the donor immune system. Removal of T-cells, however, is a labor-intensive process that increases the risks for infection and causes the loss of stem cells and facilitating cells that the donated bone marrow needs to be able to survive in its new host.

Some experimental organ transplantation treatments have attempted a two step process in patients with myeloma. The process involved inducing bone marrow transplantation from a living donor to establish chimerism and then following with transplant of the organ several weeks later; unfortunately, this process had a high risk of damage to the transplanted organ. Further, persons that are waiting for organ transplants are usually very ill, so the time between organ transplantation can be crucial. The extra time increases medical complications and cost.

Despite past attempts to achieve mixed chimerism, no consistent and safe approach has been developed for establishing mixed chimerism in a patient without significant risk of generating GVHD. For instance, approaches that deplete donor T cells from the bone marrow inoculum prior to bone marrow transplantation were intended to reduce the risk of GVHD but have also reduced the chances of successful bone marrow transplantation. These past attempts severely suppressed the bone marrow and/or immune system and caused neutropenia.

The ability to successfully establish mixed chimerism without significant risk of generating GVHD would be a major step in organ transplantation, the treatment of autoimmune diseases, cancer treatments, and pathological conditions such as hemoglobinopathies. The ability to not only reduce GVHD but also have only a small suppressive effect on bone marrow functions and immune system functions, to avoid neutropenia, and to avoid T-cell depletion steps would be another major step. The further ability to transplant bone marrow and follow with an organ or cell transplant in only a few days would represent another major step. A simultaneous bone marrow and organ transplant would be yet another major step.

### SUMMARY OF THE INVENTION

The present invention presents effective techniques and treatments for producing mixed chimerism without significant risk of generating GVHD. These techniques have only a small suppressive effect on the immune system and bone marrow functions and cause little or no neutropenia compared to other techniques. No step to treat extracted donor bone marrow to deplete T-cells is required. The techniques make it possible to introduce bone marrow and a transplanted organ or tissue within a few days of each other and, in some cases, on the same day, thereby making feasible the transplantation of organs and tissue from a non-living donor.

The techniques use the synergistic effects of a combination of reduced levels of pre-transplant immune suppression coupled with lower levels of post-transplant immune blockade. Because the techniques are generally mild in their suppression of a patient's bone marrow activity, the trauma to a patient's blood supply and immune system is minimized and the patient is able to adapt more rapidly to the infusion of donor bone marrow. Since the patient is less traumatized by the pre-treatment regimen, it is possible to decrease the amount and timing of post-transplant immune blockade therapy required to prevent GVHD. The present invention recognizes the unexpected result that these two effects actually enhance each other and are, therefore, synergistic with each other. By recognizing the synergistic effects of a combination of reduced levels of pre-transplant immune suppression coupled with lower levels of post-transplant immune blockade, the techniques of the present invention provide for treatments that rapidly induce mixed chimerism with minimal immune and hematopoietic suppression without inducing GVHD.

One treatment in accordance with a preferred embodiment of the present invention involves a conditioning step of administering fludarabine phosphate and/or cyclophosphamide prior to infusing donor bone marrow cells and blocking T-cell activity after bone marrow infusion by using agents that block or interfere with CD40 receptor/CD154 (called CD40 ligand), and CD28/B7 receptors. T-cell activity may also be blocked by Rapamycin or a comparable equivalent. MR1, 5C8, and IDEC-131 are antibody agents for blocking CD40L ligand-to-CD40 receptor interaction and CTLA4Ig is an agent that interferes with CD28-B7 receptor interaction. Since effective blocking of T-cell activity prevents GVHD, the harsh suppression of the recipient immune system and/or bone marrow cell activity that is generally favored in conventional treatments is simply not needed. Instead, only a much less toxic conditioning regimen of agents such as busulfan, fludarabine phosphate and/or cyclophosphamide is required. Because a harsh treatment of the immune system is unnecessary, mixed chimerism can be achieved more rapidly and with only a mild regimen of immune suppression. Since mixed chimerism is rapidly established, the risks of complications and unfavorable reactions are minimized.

One advantage of the techniques of the present invention is that they require only a brief inhibition of the immune function. In contrast, existing techniques for inducing mixed chimerism require a lengthy suppression of immune functions. As a result, the patient is at a much greater risk of succumbing to opportunistic maladies and must be maintained in an uncomfortable and costly hospital environment. Because the immune system is mildly inhibited by the techniques of the present invention as compared to conventional treatments, the result is that the patient's immune system recovers to normal levels more quickly and the onset of mixed chimerism is accelerated.

An advantage of the invention is that the techniques, in contrast to typical conventional techniques, do not require that donor bone marrow extracted from a donor be depleted of its T-cells. As a result, recovery and onset of mixed chimerism is accelerated. The elimination of the T-cell depletion step saves time, money, and increases reproducible and consistent results.

The techniques of the present invention also enable transplantation of organs and tissue with much less matching than conventionally practiced transplantation protocols. Mismatched donors and recipients may be used without the elaborate matching process that is conventionally required. The invention facilitates a higher degree of mismatching between donor and recipient that was previously possible and extends bone marrow and stem cell transplants to haploidentical and even completely mismatched donor-recipient pairs, including transplants from cadaveric bone marrow and peripheral blood stem cell donors.

Another advantage of the invention is that mixed chimerism establishes the graft-versus-tumor effect (GVT). The beneficial effects of GVT are difficult to separate from the detrimental effects of GVHD but these techniques prevent GVHD and promote mixed chimerism such that GVT may be achieved. Inducing GVT in a cancer patient causes their body to attack the cancer. Inducing GVT by the techniques of the present invention is a treatment for cancers.

The course of treatments may optionally include use of agents like anti-lymphocyte serum (ALS) and/or infusion of donor cells, for example spleen cells or blood cells, prior to bone marrow cell transplantation. This infusion generally enhances the establishment of mixed chimerism but is not necessary.

The techniques and treatments of the invention are applicable not only to organ transplant but also to cell transplants, treating autoimmune diseases, preventing autoimmunity and related diseases in at-risk patients and, treating cancer and other pathological conditions such as hemoglobinopathies. Indeed, this invention enables an organ transplant and bone marrow transplant to be performed simultaneously or on the same day.

### BRIEF DESCRIPTION OF THE DRAWINGS

FIG. 1 is an illustration of treatments for inducing mixed chimerism.

FIG. 2 is an illustration that compares the invention's impact on the immune system to prior art treatments.

FIG. 3 is an illustration of treatments for inducing mixed chimerism that include ALS.

FIG. 4 is an illustration of treatments for inducing mixed chimerism that include donor cell pretreatment.

FIG. 5 is an illustration of treatments for inducing mixed chimerism and transplanting tissue.

FIG. 6 is an illustration of treatments for transplanting tissue and bone marrow within 24 hours.

FIG. 7 shows how a preconditioning treatment of FL and CY reduces lymphocytes in the peripheral blood of C57BL/6 mice without reducing granulocyte and/or neutrophil populations.

FIGS. 8A and 8B show lymphocytes (R1) in mice given FL and CY conditioning treatments.

FIGS. 9A and 9B show control mice lymphocytes in the experiment of FIG. 8.

FIG. 10 shows deletion of Vβ5+ and Vβ11+ peripheral CD4+ cells in chimeric C57BL/6 Mice (at 20 Weeks Post-BMT).

FIG. 11 compares the donor specific cytokine secreting T-cells in chimeric NOD mice compared to NOD mice without Chimerism.

FIG. 12 compares PHA mitogen specific cytokine secreting T cells in chimeric and non-chimeric NOD mice.

FIG. 13 compares the onset of diabetes in chimeric and non-chimeric NOD mice.

FIG. 14 compares the survival of transplanted islets in chimeric and non-chimeric mice.

FIG. 15 shows blood glucose levels in diabetic NOD mice after simultaneous islet and bone marrow transplantation with ALS treatment, preconditioning with FL and CY, and immune blockade with Rapamycin.

FIG. 16 shows donor chimerism levels in the hematopoietic organs of mixed chimers at 20 weeks post-bone marrow transplant.

FIG. 17 is a schematic of a method of the invention for inducing mixed hematopoietic chimerism for the nonhuman primate of Example 10.

FIG. 18 is a graph of results from Example 10, showing successful induction of mixed hematopoietic chimerism in the nonhuman primate.

FIG. 19A is a schematic of an embodiment of the invention for inducing mixed hematopoietic chimerism.

FIG. 19B is a schematic of an embodiment of the invention for inducing mixed hematopoietic chimerism.

FIG. 19C is a schematic of an embodiment of the invention for inducing mixed hematopoietic chimerism.

FIG. 20 is a graph showing results for a treatment performed as depicted in FIG. 19.

FIG. 21 depicts a treatment according to an embodiment of the invention.

FIG. 22 depicts an alternative embodiment of the treatment of FIG. 22.

FIG. 23A depicts the number of islets required to treat diabetes in certain chimeric patients.

FIG. 23B depicts the number of islets required to treat diabetes in certain immunosuppressed patients.

### DETAILED DESCRIPTION OF PREFERRED EMBODIMENTS

The Immune System

A person's own immune system normally does not attack the person, a condition called self-tolerance. The immune system also has the ability to identify and respond to invading or foreign agents, an ability generally termed acquired immunity. Acquired immunity uses two main mechanisms: B-cell immunity (also termed humoral immunity) and T-cell immunity (also termed cell-mediated immunity). B-cell immunity is mediated by B-cells and involves the creation of antibodies. T-cell immunity is mediated by T-cells and involves the activation of lymphocytes that kill the foreign agents. Both T-cells and B-cells are termed lymphocytes. Both B-lymphocytes (B-cells) and T-lymphocytes (T-cells) respond when they recognize molecular-sized targets, which are called antigens. Lymphocytes have distinctive molecules on their surface that allows them to be distinguished from other cells. Once the B-cells or T-cells respond to an antigen, they begin to proliferate and send out chemical signals that cause an amplification, or cascade, of events that activate many cells and eventually causes the destruction of the foreign cells that bear the offending antigen.

There are three major groups of T-cells: two types of regulatory T-cells, termed Helper T-cells and Suppressor T-cells, and the Cytotoxic T-cells. Regulatory T-cells are helper cells that help to activate other cells in the immune system. Cytotoxic T-cells directly attack cells that have been infected by viruses or transformed by cancer and are chiefly responsible for the rejection of tissue and organ grafts. T-cells work by secreting cytokines or, more specifically, lymphokines. Lymphokines (also secreted by B cells) are chemical messengers that evoke many reactions from various cells. A single cytokine may have many functions and several cytokines may be able to produce the same effect. Many cytokines have initial names but, as their basic structure is identified, they are renamed as “interleukins” and are denoted as IL-1, IL-2, and so forth.

GVHD is thought to be mediated by T-cells in several ways. T-cells are generally active in the T-cell immunity system, so generally suppressing their functions or destroying them can counteract GVHD. Suppressing CD8-positive T-cells is an example of this approach. Another way that T-cells contribute to GVHD is by their CD40 ligand (also called CD154) on their surface binding to the CD40 receptor on dendritic or macrophage cells; since these cells “present” the antigens that are on foreign tissue, blockage of this interaction helps to prevent the T-cell immune system from attacking the foreign tissue. Another GVHD T-cell mediation mechanism involves the T-cell's CD28 ligand binding the B7 receptor (i.e., receptors termed CD80 (B7-1) or CD86 (B7-2)) on antigen-presenting cells (APCs) such as dendritic cells.

Agents for Controlling the Immune System

There is a class of drugs termed myelosuppressants that inhibit bone marrow cell function. The function of bone marrow cells includes making T-cells and hematopoiesis, which means making cells and materials required for blood to function. So generally inhibiting bone marrow cell function inhibits the function of the immune system and inhibits hematopoiesis. Another class of drugs termed immunosuppressants are more directly targeted to blocking only the immune system, for example by interfering with an important T-cell immunity receptor. Some of these immunosuppressant drugs are chemotherapy agents, which include alkaloids, alkylating agents, antimetabolites, enzymes, hormones, platinum compounds, and new drugs.

Alkylating agents are toxic chemicals that tend to react with DNA with the result that they destroy the DNA or cause it to be come crosslinked. They tend to preferentially kill proliferating cells, especially bone marrow cells and are generally myelosuppressants (inhibitors of bone marrow cell activities). Most alkylating agents can be classified as nitrogen mustards or nitrosoureas. Nitrogen mustards include mechlorethamine and chlorambucil, and melphalan; but the most commonly used alkylating agent is cyclophosphamide. It can be given in a variety of ways and dosages unlike many of the other nitrogen mustards. Ifosphamide is an alkylating agent closely related to cyclophosphamide. Nitrosoureas include carmustine, lomustine and semustine. Other alkylating agents include cyclophosphamide, busulfan, dacarbazine, hydroxymethylmelamine, thiotepa and mitocycin C.

FLUDARA is a trade name for fludarabine phosphate. Fludarabine phosphate is changed in the body to a metabolite that appears to act by inhibiting DNA polymerase alpha, ribonucleotide reductase and DNA primase, thus inhibiting DNA synthesis. It acts on a very wide range of cell types and generally stops or slows the multiplication of all cells. It is a myelosuppressant but at properly controlled levels is not myeloablative.

Cyclosporine (CSA) is an immunosuppressant that blocks gene transcription of IL-2 and other lymphokines so that T-cells do not proliferate and the immune response to a foreign antigen is suppressed. Its primary target is helper T lymphocytes, with little effect on other aspects of the immune response. CSA and tacrolimus are thought to bind to immunophilin. The CSA-immunophilin complex in turn binds to and blocks a phosphatase called calcineurin, which is needed to activate enhancers/promoters of certain genes, including those for transcription of IL-2 (and other early activation factors).

RAPAMUNE is a trade name for Sirolimus, also known as rapamycin, an immunosuppressant. Sirolimus has been shown to block T-cell activation and proliferation by blocking the response of T and B cells to cytokines, thereby preventing cell cycle progression at stage G1 and consequently blocking T-cell and B-cell proliferation. More specifically, sirolimus blocks T lymphocyte proliferation in response to IL-2 and blocks the stimulation caused by ligand binding of the T-cell's CD28 molecule. It is thought to do this by blocking activation of the kinase referred to as mammalian target of rapamycin or “mTOR”, a serine-threonine kinase that is important for cell cycle progression. It generally has synergy with cyclosporine (CSA) in vitro as well as in animal and clinical studies. It is soluble in dimethylsulfoxide (DMSO) and methanol.

Cyclophosphamide (CY) is an alkylating agent that may be used as an immune suppressant. It generally suppresses the B-cell immunity system and the T-cell immunity system by acting generally against proliferating cells. It has trade names such as CYTOXAN. As an immunosuppressant its most important effect in controlling GVT and GVHD is thought to be clonal destruction. T-cells and B-cells normally will proliferate in response to a foreign antigen so that there are many of them that respond to the same antigen; the proliferation is a key part of the immune system's amplification process. The proliferating cells are especially vulnerable to CY so that CY tends to kill all of these proliferating cells and thereby stop the amplification of the initial response to the foreign antigen. At properly controlled levels CY is not myeloablative.

Busulfan, also called Myelosan or Busulphan, is an alkylating agent that is a myelosuppressant. It has trade names such as BUSULFEX, or MYELERAN. Like other alkylating agents, it generally is believed to cross-link the DNA of proliferating cells so they die.

T-cells express a surface molecule called the CD40 ligand that binds the CD40 receptor on dendritic cells. The CD40 ligand-to-CD40 receptor binding event is important for activating T-cells to recognize a foreign antigen and for amplifying the immune response. MR1 is an agent that interferes with this binding event in mice. MR1 is an antibody against the CD40 ligand, i.e., the “antibody recognizes” or “the antibody binds” it. Other antibodies exist that also bind to the CD40 ligand or receptor in other species, for example the antibodies 5C8 and IDEC-131 that bind the CD40 ligand in humans.

Another GVHD T-cell mediation mechanism involves the T-cell's CD28 ligand binding to the B7 receptor (i.e., receptors termed CD80 (B7-1) or CD86 (B7-2)) on antigen-presenting cells (APCs) such as dendritic cells. This binding event amplifies the response of the immune system to a foreign antigen. The molecule CTLA4 (also called CD152) binds the B7 receptor so that there is not a CD28-to-B7 binding event. CTLA4 is a natural “off switch” that is present at very low concentrations in the body. REPLIGEN, Inc., manufactures CTLA4-Ig which is modeled after CTLA4 and also acts as an “off switch” by competitively inhibiting the binding of B7 to CD28. CTLA4-Ig and LEA29Y, a mutant form of CTLA4-Ig counteracts GVHD.

Tacrolimus, also called PROGRAF or FK506, is many times more potent than cyclosporine. The critical difference is that it inhibits interleukin 2 expression and synthesis, and has a specific action on T-helper lymphocytes.

Anti-lymphocyte globulin (ALG) is a mixture of antibodies against lymphocytes and acts as a general immunosuppressant. Anti-thymocyte globulin (ATG) acts in a similar fashion to ALG and is generally its equivalent. Antilymphocyte serum (ALS) is a serum of polyclonal antibodies against lymphocytes and acts in a similar fashion to ALG and is generally its equivalent.

The drugs and agents described herein are provided in a variety of forms. Some forms are preferable for a particular type of delivery such as oral, intravenous, or intramuscular. For example, BUSULFEX is a particular form of busulfan. Other forms of a drug are preferable for controlling release rates or solubility. Those skilled in these arts will immediately understand how to use the most appropriate form of the drugs or agents described herein for the particular application that is contemplated.

Medical professionals and scientists use the term myeloablative in a variety of ways. Myeloablative literally means to kill bone marrow cells, but the word is often used to describe only procedures that kill most or all of a patient's bone marrow cells. The methods described herein are nonmyeloablative in the sense that they do not kill all or most of a patient's bone marrow. These methods are mildly myeloablative in the sense that they cause the death of only a small percentage of a patient's bone marrow cells.

Neutropenia

The term neutropenia is also used in different ways. Neutropenia means a decline in the number of neutrophils, for instance in the blood or liver (Dorland's Medical Dictionary, 28th Ed.). The term neutropenia, however, can also mean a marked decline or shortage of neutrophils. The invention may cause a small decrease in neutrophils but the invention avoids neutropenia in the sense that it does not cause a marked decline or shortage of neutrophils.

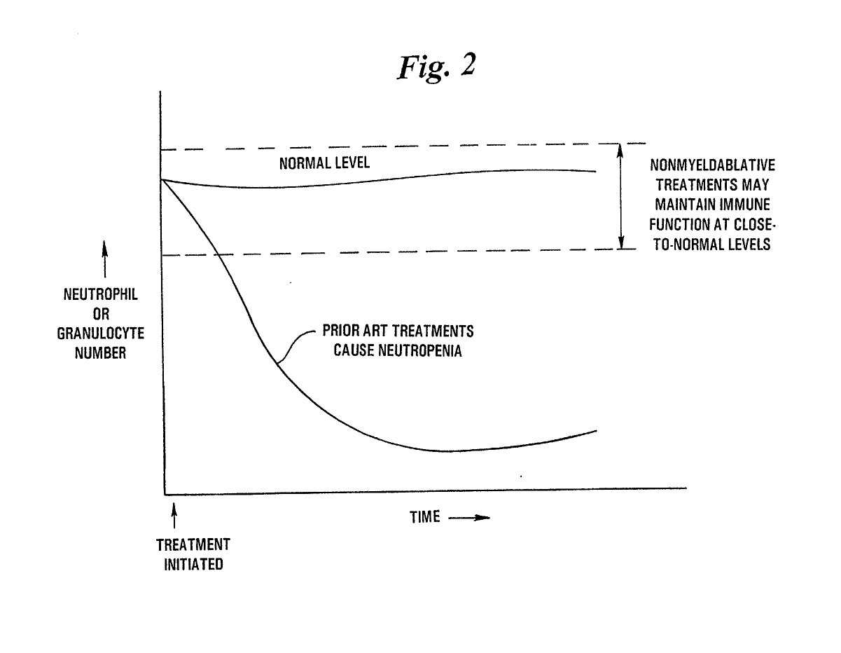

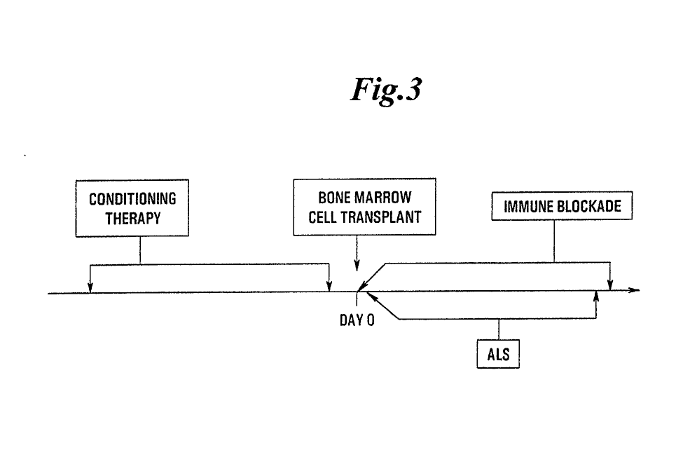

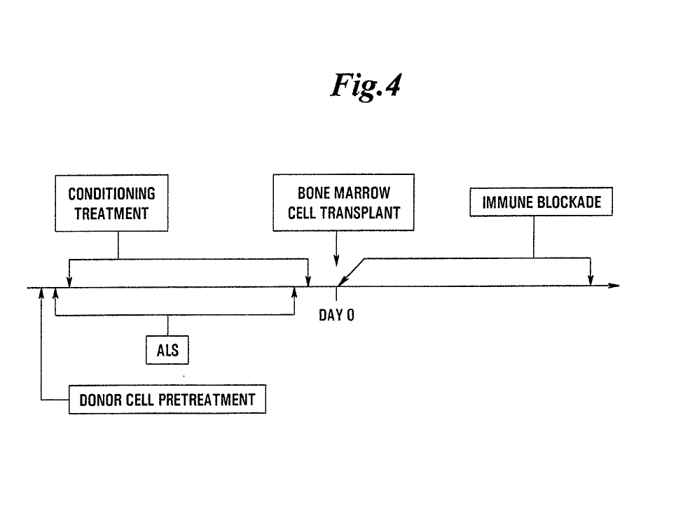

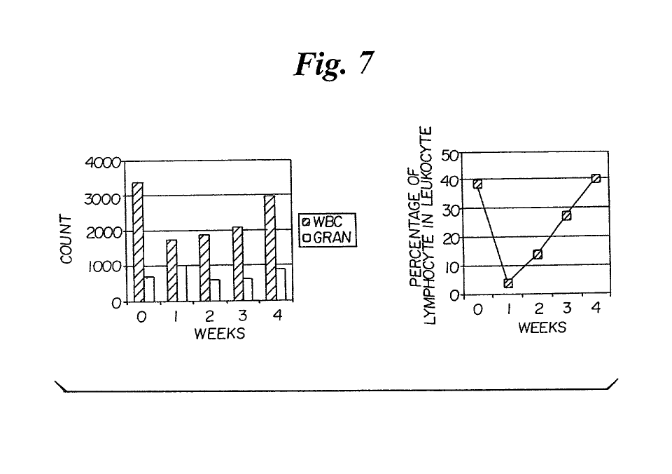

Neutrophils are a type of granulocyte, which is a white blood cell. Lymphocytes are also white blood cells. These cell types are involved in immune function. In contrast to conventional treatments, the conditioning treatment of the invention reduces the number of lymphocytes in the patient's blood but has a small impact on the number of granulocytes or neutrophils. The conditioning treatment is specifically directed to lymphocytes in the sense that it markedly and transiently decreases lymphocyte numbers (thus causing a drop on the total white blood cell count) without markedly decreasing neutrophil and/or granulocyte counts (FIGS. 2 and 7).

A measurement of the number or change in number of neutrophils or granulocytes is sufficient to indicate if a patient is suffering from neutropenia. A related condition is granulocytopenia, a condition indicated by a marked decrease in granulocytes and certain symptoms (Dorland's Medical Dictionary, 28th Ed.). One measurement that is diagnostic of neutropenia is the absolute neutrophil count (ANC), a test run on a sample of the patient's blood that is known to those skilled in these arts. An ANC of approximately 1500 to 8000 cells per μL of blood (1.5-8.0×10⁹ cell/L) is generally considered normal. An ANC of less than about 500 cell per μL (0.5×10⁹ cell/L) is generally considered neutropenic and an ANC of less than about 100 cells per 1L is generally considered to be profoundly neutropenic.

Graft Versus Host Disease (GVHD)

Current science leaves open the question of whether or not graft-versus-tumor (GVT) effects can be induced in the absence of clinically overt GVHD. Current methods that tend to promote GVT tend to also promote GVHD but suppressing GVHD tends to also suppress GVT. GVHD occurs in an early form termed acute GVHD that occurs within about the first three months following an allogeneic bone marrow cell transplant and a late form termed chronic GVHD. Acute GVHD is currently believed to be caused chiefly by the T-lymphocytes that are part of the transplanted bone marrow cell. The T-lymphocytes attack the patient's skin, liver, stomach, and/or intestines.

One approach to preventing GVHD is T-cell depletion (e.g., elutriation, monoclonal antibody treatment, and use of columns). In this approach the donor bone marrow cells are subjected to a time consuming and labor-intensive process to remove T-cells, for instance by column chromatography or separation by size and density. Removal of too many of these cells, however, will negatively impact the engraftment of donor stem cells and may prevent GVT. GVT is desired when cancer is present because it will attack the cancerous cells in the bone marrow cell recipient. This process can also cause stem cells to be lost so that additional steps to prevent the loss of the stem cells are needed, for instance by using monoclonal antibodies that recognize the stem cells. Further, important cells called facilitator cells are lost. The loss of facilitator and stem cells increases the chances that the bone marrow cell graft will not succeed, i.e., will fail to engraft.

Another approach is to use a drug such as Cyclosporine (CSA). As previously discussed, CSA is an immunosuppressive drug that suppresses the function of the donor's T-cells. For patients not receiving a T-cell depleted transplant, the use of methotrexate added to Cyclosporine may be effective in decreasing the severity of GVHD. The side effects of Methotrexate include temporary but painful mouth sores that cause difficulty in eating and swallowing and reversible liver damage.

Chronic GVHD is the late form of GVHD. It may be caused by donated bone marrow T-cells which have grown up in the patient without maturing normally. The symptoms of chronic GVHD resemble many spontaneously occurring autoimmune disorders. Chronic GVHD occurs in about 40% of patients receiving an allogeneic transplant. Treatments include the use of Thalidomide and Cyclosporine. Chronic GVHD causes the death of about 10% of all allogeneic bone marrow cell recipients.

Establishment of Mixed Chimerism and Tolerance

Mixed chimerism can induce tolerance in recipients of organs and tissues transplanted from donors. Various approaches have been used to achieve microchimerism, which is a state of the less than about 1% donor-specific antigens in the recipient, and macrochimerism, which is a state of more than about 1% donor-specific antigens in the recipient. A recipient can be chimeric in different systems of their body. For example, a transplant recipient that has a mixture of donor and recipient kidney antigens is kidney chimeric. A preferred type of chimerism is hematopoietic chimerism since the hematopoietic system makes blood and immune cells. Mixed chimerism is preferably stable but transient chimerism can also effectively create tolerance to an organ transplant.

One approach to establishing chimerism has been to expose the recipient to high levels of radiation (called total body irradiation, TBI) and then to infuse a mixture of donor and recipient bone marrow cells wherein the donor bone marrow cells have been treated to remove lymphocytes. (Sachs et al., Ann. Thorac. Surg., 56:1221 (1993); Illstad et al., Nature, 307:168 (1984)). Lower doses of TBI have also been used and followed by infusion of donor bone marrow cells plus antibodies against CD4 positive T-cells and CD8 positive T-cells and also natural killer cells to cause a general inhibition of immune function (Tomita et al., Transplantation, 61:469 (1996)). Others have used TBI plus a very high number of donor-derived hematopoietic cells that have been depleted of T-cells (Reisner et al., Immunol. Today 16:437 (1995); Bachar-Lustig et al., Nature Medicine, 12:1268 (1986)). TBI plus CY has also been reported.

Another approach is total lymphoid irradiation (TLI). In this approach, high doses of radiation (3,400-4,440 Gy) are used followed by infusion with donor bone marrow cells. TLI strongly suppresses the immune system. TLI reduces exposure of the recipient's bone marrow cell. This technique involves large amounts of radiation, repeated and lengthy in-clinic treatment, and has significant side effects.

Other variations of TLI and TBI treatments have been reported, for example, by Slavin and colleagues (PCT Publication No. WO 00/40701 A3, filed Dec. 23, 1999). Ildstad (U.S. Pat. No. 5,876,692) reports that anti-lymphocyte globulin (ALG) may be used to decrease the amount of TBI or TLI dosage. Other toleration protocols have been claimed, such as by Sachs in U.S. Pat. No. 5,876,708 wherein hematopoietic stem cells are introduced into a recipient, the recipient's T-cells are inactivated, the patient is immunosuppressed without recourse to antibodies against T-cells, and the recipient receives a graft from the donor. Other protocols claimed are, for instance, by Sykes in U.S. Pat. No. 6,006,752, which has claims to the creation of thymic space by irradiation or certain drug combinations.

One attempt to balance GVT with GVHD has been to infuse donor lymphocytes (DLI) into a recipient in incremental steps so as to provoke GVT and stop infusions after GVHD become too severe or difficult to control (Morecki and Slavin, J. Hematotherapy & Stem Cell Res 9:355, 357 (2000)). DLI has been performed before and after transplants but continues to carry significant risk of graft rejection or life-threatening GVHD. The need to balance GVT against GVHD is shown, for instance, in the attempt to promote GVT in a man that resulted in his death by GVHD (PCT Publication No. WO 00/40701 A3, Example 16).

Another attempted approach involves T-cell depletion, which is associated with a decrease in the risks of GVHD. Studies in rodents show that depleting T-cells can avoid GVHD risks (see Reich-Zeliger et al., Immunity 13:507-515, 2000). This procedure, however, is time-consuming, labor-intensive, requires multiple patient visits, and is often associated with the failure of bone marrow cells to engraft.

In animal models, it has been demonstrated that allogeneic bone marrow cell transplantation is a powerful treatment for various autoimmune diseases. However, the clinical application of bone marrow cell transplantation for nonmalignant diseases has been extremely limited, because these approaches largely rely on irradiation and treatments that severely suppress the immune and/or hematopoietic systems. These approaches are too toxic for widespread use in humans.

Bone marrow cell transplantation with such protocols induced either full chimerism or mixed chimerism in preconditioned hosts. In the setting of organ tissue transplants and autoimmune disease, low levels of stable donor mixed chimerism may be adequate to induce tolerance and continue autoreactivity. An early study by Cobbold et al., demonstrated that allogeneic bone marrow cell engraftment and specific tolerance could be achieved by a sublethal dose of total body irradiation and treatment of deleting anti-CD4 and anti-CD8 monoclonal antibodies. Subsequently, mixed chimerism as an approach for inducing tolerance in small animal models was extensively investigated using irradiation as a conditioning therapy. See Mixed Chimerism as an Approach for the Induction of Transplantation Tolerance, T. Wekerle and M. Sykes, Transplantation 68:459-467, 1999; and Mixed Chimerism as an Approach to Transplantation Tolerance, D. H. Sachs, Clinical Immunol. 95: S63-S68, 2000.

Recent studies report that mixed chimerism could also be induced by using costimulatory blockade and high-dose bone marrow cell transplantation (See Allogeneic Bone Marrow Transplantation With Co-Stimulatory Blockade Induces Macrochimerism and Tolerance Without Cytoreductive Host Treatment, T. Wekerle, J. Kurtz, H. Ito, J. V. Ronquillo, V. Dong, G. Zhao, J. Shaffer, M. H. Sayegh, and M. Sykes, Nat. Med. 6:464-469, 2000) or repeated bone marrow cell transplants. See Cutting Edge Administration of Anti-CD40 Ligand and Donor Bone Marrow Leads to Hemopoietic Chimerism and Donor-Specific Tolerance Without Cytoreductive Conditioning, M. M. Durham, A. W. Bingaman, A. B. Adams, J. Ha, S. Y. Waitze, T. C. Pearson, and C. P. Larsen, J. Immunol. 165:1-4, 2000. Hale et al., also reported that stable mixed chimerism can be established by a high dose of bone marrow cell, anti-lymphocyte serum (ALS), and rapamycin treatment. See Establishment of Stable Multilineage Hematopoietic Chimerism and Donor-Specific Tolerance Without Irradiation, D. A. Hale, R. Gottschalk, A. Umemura, T. Maki, and A. P. Monaco, Transplantation 69:1242-1251, 2000. However, these protocols are difficult to apply clinically because of the total amount of bone marrow cell required for transplantation. With a small amount of bone marrow cell, Tomita et al., showed that mixed chimerism could be induced in fully MHC-mismatched mice after donor spleen cell pretreatment followed myelosuppressive busulfan and cyclophosphamide. See Induction of Permanent Mixed Chimerism and Skin Allograft Tolerance Across Fully MHC-Mismatched Barriers by the Additional Myelosuppressive Treatment in Mice Primed With Allogeneic Spleen Cells Followed by Cyclophosphamide, Y. Tomita, M. Yoshikawa, Q. W. Zhang, I. Shimizu, S. Okano, T. Iwai, H. Yasui, and K. Nomoto, J. Immunol. 165:34-41, 2000.

Mixed Chimerism Established by the Present Invention

Chemically induced mouse diabetic models have generally been used for islet transplantation and immune tolerance. However, they cannot truly reflect the clinical setting of autoimmune diabetes. It is for this reason that the NOD mouse has been extensively used as an animal model of human type 1 diabetes and is a scientifically accepted model for autoimmune diabetes. The development of diabetes in these mice has been attributed to autoreactive T-cells that infiltrate pancreatic islets and specifically destroy insulin-producing islet beta cells. Islet allografts in diabetic NOD mice are destroyed by both alloimmune and recurrent T-cell-mediated anti-islet autoimmune responses (allograft means a graft from another individual of the same species; alloimmune means the immune system of another individual of the same species). The NOD mouse model is the best available model for experimental islet transplant research and predictive for the development of clinically relevant methods to induce and restore tolerance in humans.

A multitude of strategies have been shown to prevent the development of diabetes in NOD mice. Sublethal irradiation is one approach proven to prevent graft rejection and autoimmune destruction of islet allografts in overtly diabetic NOD mice. This approach establishes mixed allogeneic chimerism that simultaneously induces donor-specific tolerance to islet allografts and restores self-tolerance to islet autoantigens. See Allogeneic Chimerism Induces Donor-Specific Tolerance to Simultaneous Islet Allografts in Non-Obese Diabetic Mice, H. Li, C. L. Kaufman, and S. T. Ildstad, Surgery 118:192-197, 1995; and Allogeneic Hematopoietic Chimerism in Mice Treated With Sublethal Myeloablation and Anti-CD154 Antibody; Absence of Graft-versus-Host Disease, Induction of Skin Allograft Tolerance, and Prevention of Recurrent Autoimmunity in Islet-Allografted NOD/Lt Mice, E. Seung, N. Iwakoshi, B. A. Woda, T. G. Markees, J. P. Mordes, A. A. Rossini, and D. L. Greiner, Blood 95:2175-2182, 2000. Since NOD mice are irradiation-resistant, a high dose of irradiation is required to establish mixed chimerism, compared with other mouse strains. See Patterns of Hemopoietic Reconstitution in Non-Obese Diabetic Mice: Dichotomy of Allogeneic Resistance Versus Competitive Advantage of Disease-Resistant Marrow, C. L. Kaufman, H. Li, and S. T. Ildstad, J. Immunol. 158:2435-2442, 1997. Such high-doses of irradiation, however, are unacceptable for the establishment of mixed chimerism in patients with diabetes. Indeed, it has proved extremely difficult to prevent rejection, to prevent autoimmune destruction, and to induce tolerance in overtly diabetic NOD mouse recipients (Immunosuppression Preventing Concordant Xenogeneic Islet Graft Rejection is not Sufficient to Prevent Recurrence of Autoimmune Diabetes in Non-Obese Diabetic Mice, Z. Guo, D. Mital, J. Shen, A. S. Chong, Y. Tian, P. Foster, H. Sankary, L. McChesney, S. C. Jensik, and J. W. Williams, Transplantation 65:1310-1314, 1998). See NOD Mice Have a Generalized Defect in Their Response to Transplantation Tolerance Induction Diabetes, T. G. Markees, D. V. Serreze, N. E. Phillips, C. H. Sorli, E. J. Gordon, L. D. Shultz, R. J. Noelle, B. A. Woda, D. L. Greiner, J. P. Mordes, and A. A. Rossini, Diabetes 48:967-974, 1999, and Immunotherapy With Nondepleting Anti-CD4 Monoclonal Antibodies but not CD28 Antagonists Protect Islet Graft in Spontaneously Diabetic NOD Mice From Autoimmune Destruction, Allogeneic and Xenogeneic Rejection, Z. Guo, T. Wu, N. Kirchof, D. Mital, J. W. Williams, M. Azuma, D. E. R. Sutherland, and B. J. Hering, Transplantation, 71:1656-1665, 2001.

The preferred embodiment of the present invention includes a system of treatments for establishing mixed chimerism in mammals using a nonmyeloablative approach. An optional treatment is donor cell pretreatment, which enhances the induction of mixed chimerism. Treatment with donor cell antigens is an example of donor cell pretreatment. The cells may be living, viable cells or nonliving cells or cell fragments. Antigens from tissue sources other than cells may also be used in this role. Pretreatment by donor spleen cells is an example of donor cell pretreatment and donor antigen pretreatment. The treatments are based on an appreciation of the function of the immune system and the function of medicinal tools that are used to control the immune system. The treatments, however, do not necessarily rely on any one particular theory of how the immune system or these medical tools function.

Mixed chimerism may be used to treat autoimmune diseases, including diabetes. Establishing mixed chimerism with the procedures of the invention prevents the onset of diabetes. Mixed chimerism probably favors migration of donor-derived cells to the recipient's thymus, where presentation of autoantigens by donor-derived antigen-presenting cells overcomes defective negative thymic selection of autoreactive T cells. As a result, autoreactive T cells undergo apoptosis in the thymus before appearing in the peripheral circulation. In addition, other mechanisms involving deletional and regulatory pathways are theorized to be involved in the restoration of self-tolerance.

The development of safe and effective methods for establishing chimerism across MHC barriers is of paramount importance for the design of treatment strategies in transplantation, autoimmunity, hematology, and oncology. Several observations and factors contribute to the present system of treatments, and are reviewed in U.S. patent application Ser. No. 09/855,027 entitled “Systems and Methods for Inducing Mixed Chimerism”, which is hereby incorporated herein by reference, which includes a discussion of Fludarabine phosphate (FL), cyclophosphamide (CY), and immune blockade reactions, e.g., CD40/CD154 interactions.

Various approaches for hematopoietic cell transplants for the treatment of hematological malignancies have recently been developed (1) based on the paradigm that aggressive conditioning therapy is not needed to achieve alloengraftment or to eradicate malignancy. In a recent multicenter trial, more than 50% of eligible patients received hematopoietic cell transplants entirely on an outpatient basis, however application of hematopoietic cell transplants for the treatment of nonmalignant conditions has remained impeded by the by the formidable problem of GVHD encountered in approximately 50% of the recipients of “minitransplants.(14) To treat hematologic malignancies, complete hematopoietic reconstitution by bone marrow transplant is conventionally considered advantageous in order to assure elimination of the malignancy.

For the purpose of inducing tolerance to an allograft or restoring self-tolerance in autoimmunity, however, it is neither necessary nor desirable to fully replace the host's hematopoietic system. Instead, the establishment of allogeneic mixed chimerism (i.e., survival of both host and donor hematopoietic elements) has several advantages, especially for hematopoietic cell transplants across MHC barriers. It can successfully achieve tolerance while otherwise maintaining full immunocompetence in mice, (15;16) miniature swine,(17) and filly mismatched cynomolgus monkeys.(18) Nevertheless, the nonmyeloablative conditioning regimens applied until recently for the induction of mixed chimerism have required exhaustive T-cell depletion to eliminate preexisting mature donor-reactive T cells; splenectomy in NHP recipients to prevent alloantibody formation, thymic irradiation to overcome intrathymic alloresistance; and myelosuppression to create “space” in the recipient's immune system. Considerable advances of the understanding of the critical events that control alloengraftment of hematopoietic cells have led to the development of novel mixed chimerism induction protocols with reduced toxicity.(2;3;5;6;8;19) Three areas of progress are especially pertinent.

First, both costimulatory blockade (2-9) and sirolimus (11;12) therapy have been found to be effective in promoting hematopoietic cell transplants alloengraftment and in preventing GVHD without the need for thymic irradiation and exhaustive host T-cell depletion (previously required to facilitate engraftment across MHC barriers). Of note, anti-CD40L mAb administration provided alloengraftment effects in murine models equivalent to 450-500 cGy TBI.(5) It had been hypothesized that the administration of costimulatory blockade at the time of donor hematopoietic cell infusion causes a profound reduction of the donor-reactive T-cell clone size in the periphery, yet leaves the remaining repertoire essentially intact by mechanisms that share characteristics of both activation-induced cell death and passive cell death.(20;21) This extrathymic deletion of alloreactive T cells permits development of macrochimerism, which then maintains tolerance through intrathymic deletion.(3) However, more recent studies have demonstrated that tolerance to the donor is established before peripheral deletion of donor-reactive T cells is complete, suggesting that nondeletional peripheral mechanisms are also operative in the initial induction of tolerance of peripheral donor-reactive T cells.(21) Recent work indicates that host CD4+ cells are required for the induction of donor-specific tolerance by anti-CD40L mAb. (5) CD4+ cell depletion, but not coating of CD4+ T cells, abrogated engraftment induced by anti-CD40L mAb. These data are consistent with the hypothesis that a CD4+ regulatory T-cell is induced by anti-CD40L mAb.(5) Additional studies revealed a fundamental role for CD4+ CD25+ cells in the induction of tolerance to alloantigen and suggested that CD4+ CD25+ cells may be vital to tolerance induction to alloantigen in strategies involving T-cell costimulation blockade.(22) Therefore, nonmyeloablative strategies based on anti-CD40L mAb therapy that preserve CD4+ CD25+ regulatory cell function may prove to be particularly successful in inducing mixed chimerism. Another group of scientists recently published preliminary data on the efficacy of anti-CD40L mAb therapy in a non-human primate (NHP) mixed chimerism model. By adding two doses of anti-CD40L mAb to a mixed chimerism induction protocol in NHPs, recipients developed significantly higher, more prolonged chimerism, yet this short course of costimulatory blockade was not sufficient to prevent alloantibody production, overcome thymic alloresistance, and induce stable mixed chimerism.(23) To completely remove thymic irradiation from the regimen and to overcome alloantibody induction, longer blockade of the CD40:CD40L pathway or concomitant use of other costimulation-blocking agents was felt to be necessary.(23) Data obtained by the Applicants in the NHP primate model and reported herein indicate that prolonged therapy with the anti-CD40L mAb IDEC-131, in combination with 200 cGy TBI, rapamycin, and CsA, successfully established transient chimerism and is sufficient to achieve tolerance.

Second, alloengraftment of donor hematopoietic cells in the complete absence of cytoreductive conditioning, i.e. without any myelosuppression, has been achieved by high-dose hematopoietic cell infusion under the cover of combined costimulatory blockade (24) or by repeated infusions of donor bone marrow and anti-CD40L mAb in the murine model,(6) thus further reducing the toxicity of the mixed chimerism approach. High-dose PBSC transplantation facilitated the induction of lasting mixed chimerism across both minor and major histocompatibility barriers in the preclinical large animal pig model in the absence of TBI.(25) These data are consistent with the possibility that niches for the engraftment of administered stem cells are filled in the absence of TBI by mass action through the infusion of a “megadose” of hematopoietic stem cells. The fact that cells within the CD34+ compartment lack B7 and are endowed with potent veto activity (26;27) is believed to be an additional important mechanism facilitating alloengraftment.(28) A megadose HCT has also proven effective in overcoming one-haplotype-mismatched MHC barriers in related, myeloablated recipients with high-risk leukemia.(29) These data suggest that a megadose hematopoietic cell transplants may also facilitate transgressing MHC barriers under nonmyeloablative conditions, thereby making the approach of mixed hematopoietic chimerism more widely available to the fields of transplantation, autoimmunity, hematology, and oncology.

Third, intraportal administration of donor cellular antigen is an emerging strategy to prevent early rejection and promote engraftment of subsequent intravenous same-donor hematopoietic cell infusions. Administration of antigens orally or through the portal vein has long been recognized to be less immunogenic.(30) A scientific group demonstrated persistent donor-specific tolerance of full-thickness skin transplants across major and minor histocompatibility barriers in mice given portal venous, followed by intravenous, infusion of same-donor hematopoietic cells.(31) More recently, the same group extended these findings to the pig skin allotransplant model.(32) When combined with fractionated irradiation, portal venous, followed by intravenous, infusion of bone marrow cells completely ameliorated intractable autoimmune disease in mice.(33) Preliminary evidence of donor-specific hyporesponsiveness after intraportal and subsequent intravenous infusion of high-dose PBSCs has recently been demonstrated by Trivedi et al. in living related kidney allograft recipients.(34)

These studies underscore the Applicant's approach, which establishes a correct use of anti-CD40L mAb. The anti-CD40L is preferably combined with sirolimus, CsA, thymic irradiation, and/or donor cellular antigen administered peripherally or intraportally. Thus the subsequent induction of stable intrathymic donor T cell chimerism without the need for profound T-cell depletion and/or splenectomy may be achieved by: (i) initial contraction of the alloreactive T cell clone size, (ii) activation of CD430 CD2530 regulatory cells in the periphery, and/or (iii) control of intrathymic alloresistance (5;21;22;35) Despite significant interest in tolerance, its achievement in the clinical setting has previously remained elusive.(37;38) Traditionally, tolerance approaches have been categorized as peripheral, involving deletion, anergy, regulation, or a combination of all three, or as central thymic deletional.(37) Progress in peripheral tolerance has been limited by the incomplete understanding of the mechanisms involved, by the lack of validated markers of the tolerant state, and by the absence of compelling preclinical data. Conversely, central deletional tolerance following hematopoietic cell transplants is mechanistically well-understood, robust, stable, compatible with transient immunosuppression, effective in restoring self-tolerance in autoimmunity, measurable (mixed chimerism is a reliable marker of central tolerance), and thus clinically applicable, provided compelling evidence of the safety and efficacy of a specific strategy is obtained in relevant NHP studies.(37) Indeed, achieving effective transplantation tolerance is a crucial goal in the effort to reduce long-term morbidity and mortality in solid organ and cellular transplant recipients.(20)

While mixed chimerism protocols are applicable for the treatment of a number of conditions, a preferred embodiment of the invention is living donor islet (and solid organ) transplantation. This embodiment has been tested in the relevant preclinical NHP model, see, e.g., Examples 9-11 The donor-specific immunologic tolerance protocols described as embodiments of the invention herein avoid both acute and chronic graft rejection as well as the side effects, inconvenience, and costs associated with chronic, nonspecific immunosuppressive therapy.

Induction of Mixed Chimerism

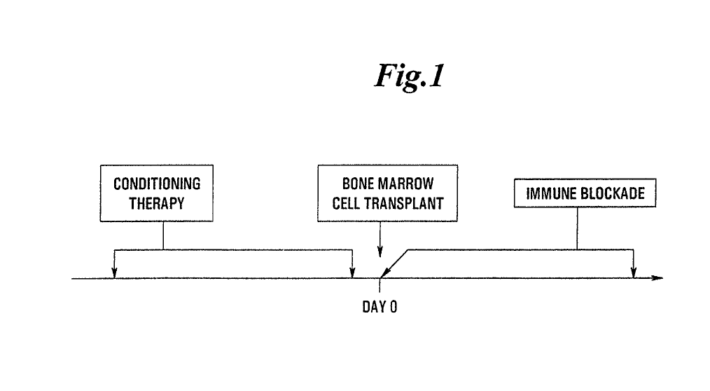

Mixed chimerism may be induced according to the present invention by performing a conditioning treatment, a bone marrow transplant, and an immune blockade (FIG. 1). The conditioning treatment mildly suppresses the immune system so that the transplanted bone marrow is not immediately rejected. The conditioning treatment avoids neutropenia and is only mildly myeloablative. The conditioning treatment prepares the recipient to receive the donor bone marrow. The bone marrow transplant involves taking bone marrow, stem cells, hematopoietic cells, immune system cells, or a combination of such cells from a donor and transplanting them into the recipient. Bone marrow transplantation may be performed in one medical procedure or in a series of smaller steps. Immune blockade prevents GVHD and enhances induction of mixed chimerism. It prevents the immune systems from attacking each other until they are fully integrated.

Conditioning Treatment

The conditioning treatment of the invention suppresses the recipient's immune system but avoids neutropenia and is nonmyeloablative or mildly myeloablative. In contrast, conventional conditioning treatments often cause neutropenia and are not mildly myeloablative. Some current publications describe certain irradiation treatments as nonmyeloablative but such treatments are not nonmyeloablative in the sense that the invention is nonmyeloablative because the irradiation treatments destroy a large percentage of the patient's bone marrow cells and a substantially higher percentage than the treatments of the invention. In an alternate embodiment, other conditioning treatments that avoid neutropenia and are only mildly myeloablative may be used; for example, a regimen of irradiation administered at doses significantly less than practiced in many conventional conditioning treatments.

In one preferred embodiment of the invention uses FL and CY in combination for the conditioning therapy. Other combinations include busulfan alone or in combination with one or both of FL and CY. FL can be replaced by other purine nucleoside analogs, such as deoxycoformycin and 2-chloro-2′-deoxyadenosine and drugs with activity against dividing or non-dividing lymphocytes. CY may be replaced by other agents that may be used nomnyeloablatively such as ifosfamide, etoposide, mitoxantrone, doxorubicin, cisplatin, carboplatin, cytarabine, and paclitaxel. Low doses of drugs conventionally used or referred to as myeloablative drugs can be used in appropriate doses, such as nitrosoureas, melphalan, thiotepa, total body irradiation, and total lymphatic irradiation.

The conditioning treatment is preferably started and concluded when the bone marrow transplant is performed (FIG. 1). This timing is preferred because the immunosuppressive effect of the conditioning treatment prepares the recipient's immune system to cooperate with the donor immune system instead of attacking it. Thus, starting the conditioning treatment after the transplant is less preferred. The conditioning treatment may be started less than 48 hours before the bone marrow transplant. Preferably, the conditioning treatment is started less than two weeks and optimally less than five days before the bone marrow transplant.

Bone Marrow Transplant

Bone marrow transplants may be performed in numerous ways known to those skilled in these arts. A common technique is to extract bone marrow from a donor's bones. The bone marrow may then be treated in a variety of ways; for example, the stem cells may be extracted and the bone marrow transplant accomplished by transplanting the stem cells to the recipient. Alternatively, stem cells may be recovered from a donor by other means, for example from their peripheral blood. The provision of stem cells may be performed according to techniques known to those skilled in these arts, for example, as described in Weissman I L, Anderson D J, Gage F., “Stem and progenitor cells: origins, phenotypes, lineage commitments, and transdifferentiations”, Annu Rev Cell Dev Biol. 2001;17:387-403; Weissman I L, “Stem cells: Units of development, units of regeneration, and units in evolution”, Cell. 100(1):157-168, Jan. 7, 2000 ; Murray L J. Tsukamoto A. Hoffman R. “Cd3430 Thy-1+Lin- Stem Cells From Mobilized Peripheral Blood”, Leukemia & Lymphoma. 22(1-2):37 ff., 1996 Jun; Peschle C, Botta R, Muller R, Valtieri M, Ziegler B L, “Purification and functional assay of pluripotent hematopoietic stem cells”, Rev Clin Exp Hematol. 2001 Mar;5(1):3-14; Peshavaria M, Pang K. Related Articles, “Manipulation of pancreatic stem cells for cell replacement therapy.”, Diabetes Technol Ther. 2000 Autunm;2(3):453-60; Moore J, Brooks P. “Stem cell transplantation for autoimmune diseases”, Springer Semin Immunopathol. 2001;23(1-2):193-213; Zandstra P W, Nagy A, “Stem cell bioengineering”, Annu Rev Biomed Eng. 2001;3:275-305; Sukhikh G T, Malaitsev V V. “Neural stem cell: biology and prospects of neurotransplantation”, Bull Exp Biol Med. 2001 Mar;131(3):203-12; Suzuki A, Nakano T., “Development of hematopoictic cells from embryonic stem cells”, Int J Hematol. 2001 Jan;73(1):1-5; Peck AB, Chaudhari M, Cornelius J G, Ramiya V K., “Pancreatic stem cells: building blocks for a better surrogate islet to treat type 1 diabetes”, Ann Med. 2001 Apr;33(3):186-92; and, Weisdorf D J, Verfaille C M, Miller W J, Blazar B R, Perry E, Shu X O, Daniels K, Hannan P, Ramsay N K, Kersey J H, McGlave P B., “Autologous bone marrow versus non-mobilized peripheral blood stem cell transplantation for lymphoid malignancies: a prospective, comparative trial”, Am J Hematol. 1997 Mar;54(3):202-8 which references are hereby incorporated herein by reference. The stem cells may be hematopoietic stem cells or stem cells that are sufficiently plastic to differentiate into pluripotent cells and specialized cells of the immunologic and hematopoietic systems. The methods herein may be used with a human donor and also with a non-human, for example, a pig or primate.

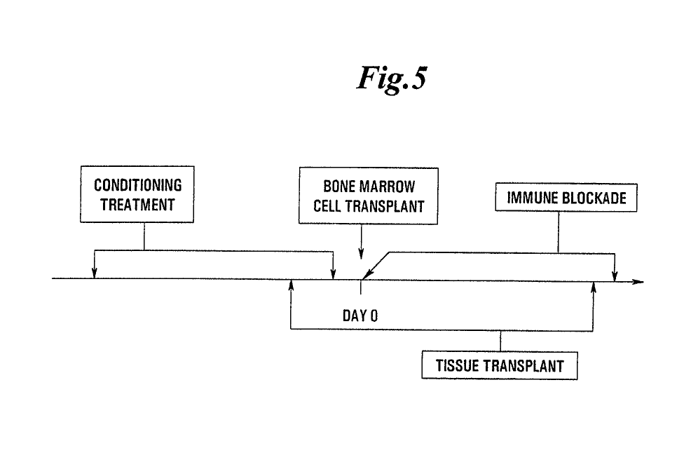

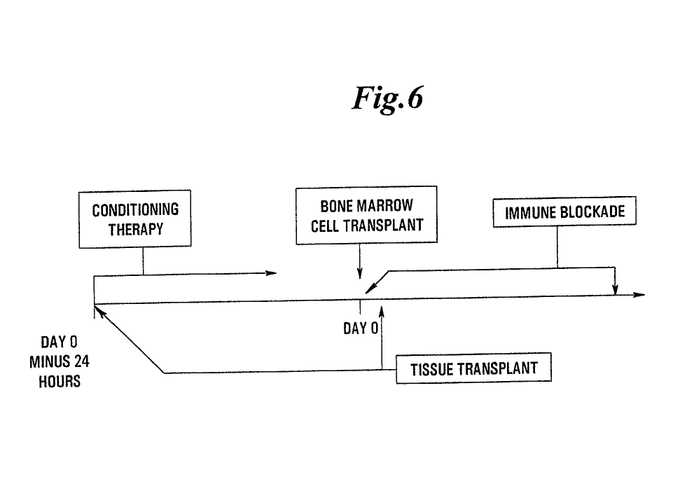

The bone marrow cell dosage and time of infusion may be varied, for example a modest dose of bone marrow may be infused several days before or after tissue transplantation (FIG. 5). The bone marrow transplant is preferably performed after the conditioning treatment has begun because it is desirable to at least mildly suppress the immune system to protect the transplanted cells. It is possible to overlap the beginning of bone marrow transplants with the end of conditioning therapy.

Immune System Blockade

The immune system blockade is preferably performed by use of agents that specifically suppress lymphocytes, preferably T-cells. Immune system blockade may include agents that block the T-cell co-stimulatory pathways, e.g., CTLA4Ig/LEA29Y or anti-CD154 (also called anti-CD40L). Another preferred embodiment of the invention uses agents that block the response of T-cells to cytokines, e.g., rapamycin. Rapamycin may be replaced by immunosuppressants such as corticosteroids, methotrexate, cyclosporins, tacrolimus, mycophenolate mofetil, leflunomide, and FTY720. Thus immune blockade is an immunotherapeutic intervention that results in, or controls and limits alloreactive T-cell function. While it is preferable to suppress the CD40:CD40L pathway, other immune blockade pathways may also be suppressed alternatively or in combination with the CD40:CD40L pathway, e.g., as described in Watts T H, DeBenedette M A, “T cell co-stimulatory molecules other than CD28”, Curr Opin Immunol 1999;1 1:286-293; Lens S M A, Tesselaar K, van Oers M H, van Lier R A, “Control of lymphocytes function through CD27-CD70 interactions”, Seminar Immunology 1998;10:491-499; Weinberg A D, Vella A T, Croft M: OX-40, “Life beyond the effect T cell stage”, Seminar Immunology 1998;10:471-480; Vinay D S, Kwon B S, “Role of 4-1BB in immune response,” Seminar Immunology 1998;10:481-489; Tikkanen J M, Lemstrom K B, Koskinen P K. “Blockade of CD28/B7-2 costimulation inhibits experimental obliterative bronchiolitis in rat tracheal allografts: suppression of helper T cell type1-dominated immune response”, Am J Respir Crit Care Med. 2002;165(5):724-9; and, Suzuki A, Satoh S, Tsuchiya N, Kato T, Sato M, Senoo H., “[Upregulation of costimulatory adhesion molecule (CD80) in rat kidney with ischemia/reperfusion injury]”, Nippon Hinyokika Gakkai Zasshi, 2002 93(1):33-8, which are hereby incorporated by reference herein.

The immune system blockade of the invention is used to prevent GVHD and to enhance chimerism. Since the blockade suppresses the activity of the donor cells it is preferable to begin the blockade at approximately the same time as the donor bone marrow is administered (FIG. 1). The use of immune blockade prior to transplant is possible but is inefficient.

Administration of Anti-Lymphocyte Serum (ALS)

The use of ALS is optional and is intended to enhance the induction of mixed chimerism ALS is specific to lymphocytes and suppresses the activity of host and donor immune systems. ALS is believed to enhance mixed chimerism by generally suppressing the immune systems and destroying clones of lymphocytes that react to the host or to the donor. Therefore, it is preferable to add ALS approximately when donor cells are introduced for the first time, either in the form of bone marrow cells or cells used for the cell pretreatment step. ALG, ATG, anti-CD3 mAb (OKT3), anti-CD4, and anti-CD8 are agents that may be used to replace ALS.

Rapamycin is preferably used in combination with the ALS treatment or its equivalent. The use of ALS and/or rapamycin may be replaced by costimulatory blockades such as anti-CD154 mAb, CTLA4Ig or anticytokine agents, for example anti-tumor necrosis factor, or regulatory cytokines, for example transforming growth factor beta or IL-10.

Donor Cell Pretreatment in Combination with ALS

Donor cell pretreatment is optional and may be used to enhance the induction of mixed chimerism. Donor cells are cells that display antigens to the recipient immune system that are given to the recipient prior to the bone marrow transplant. Spleen cells are useful donor cells but blood or cells taken from blood are also effective. The mechanism of the enhancement of chimerism is believed to be that the pretreatment cells trigger the recipient's immune system to begin to train lymphocytes and to amplify its response against the donor cells. Once this process is triggered, agents such as ALS may be added that partially destroy the recipient immune system's capability to respond to the donor cells. Donor cell pretreatment is preferably started prior to the infusion of immune system cells.

Donor Tissue Transplantation

Donor tissue transplants may be performed in numerous ways known to those skilled in these arts. The donated tissue is preferably transplanted 48 hours before or after the bone marrow transplantation so that tissue donation from a brain-dead organ donor (cadaveric donor) may readily be accomplished. A longer time period begins to introduce complications stemming from storage of the donor tissue. Alternatively, the bone marrow cell transplantation may be spread out into a number of doses over a time course or the donated tissue may be transplanted many days after the bone marrow cell transplantation.

The methods and systems of the present invention for producing mixed chimerism are effective for producing tolerance to any donated tissues. For example, tolerance may be induced that will allow safe transplantation of organs or tissues such as kidneys, livers, hearts, lungs, pancreas, small bowel, skin, neurons, and hepatocytes. Further, it is not necessary to limit transplantation to HLA-matched (MHC-matched) donors and recipients. Mismatches of more than 2 HLAs (2 MHC antigens) are possible.

### EXAMPLES

Many aspects of the protocols and procedures are familiar to those skilled in these arts and are described in contemporary literature. The day of bone marrow cell transplantation is sometimes referred to as day 0, abbreviated do; similarly 2 days before is d-2 and 2 days after is d2.

Certain elements found in the Examples have been described elsewhere. Such descriptions, however, do not set forth the systems, methods, and combinations of the present invention. Mixed chimerism is a platform strategy to i) permit allogeneic cell and solid organ transplantation without the need for chronic immunosuppression, ii) control autoreactivity in autoimmune disorders, iii) alleviate clinical symptoms in hemoglobinopathies, genetically based immunodeficiencies and enzyme deficiencies, and iv) achieve full chimerism with reduced risk of graft-versus-host-disease (GVHD) through subsequent same-donor lymphocyte infusions for the treatment of hematological and selective non-hematological malignancies. Patents and patent applications that provide further details relevant to some procedures found in the present application are: PCT/US99/02443; PCT/US97/20946; PCT/US99/30704; PCT/US98/02141; PCT/US00/02910; PCT/US98/24209; PCT/US99/02443; PCT/US97/07874; PCT/US97/07874; WO9839427 and U.S. Pat. Nos. 5,876,692, 5,665,350, and 6,068,836 which are hereby incorporated by reference, as are all patents, patent applications, and other references cited in this Application.

### Example 1

This example shows that donor cell pretreatment enhances the induction of allogeneic mixed hematopoietic chimerism in C57BL/6 and NOD mice when using nonirradiative and nonmyeloablative approaches. Allogeneic mixed hematopoietic chimerism can be used as an approach for inducting tolerance to alloantigens and restoring self-tolerance to autoantigens for islet transplantation. However, toxicity of conditioning therapy and the complication of bone marrow engraftment currently limits its clinical application. The NOD mouse strain, which is a mouse model of human type 1 diabetes, is irradiation-resistant and using conventional treatments, a high dose of irradiation has to be given in order to achieve mixed chimerism. The nonirradiative and nonmyeloablative fludarabine based conditioning therapies herein, however, produce sufficient immunosuppression to allow engraftment of allogeneic bone marrow cells. Anti-CD40 monoclonal antibody and rapamycin have been used to prevent the GVHD. This study showed that allogeneic mixed chimerism can be induced in C57BL/6 mouse strain and NOD mouse strain after transplantation of a modest bone marrow dose by using nonirradiative and nonmyeloablative fludarabine based approaches and that donor cell pretreatment enhances the induction of mixed chimerism. Balb/c spleen cells (H-2^{d}, 1×10⁸) were given intravenously (i.v.) at day −3 before bone marrow transplantation. Fludarabine (FL, 400 mg/kg) and cyclophosphamide (CY, 200 mg/kg) was given intraperitoneally (i.p.) at day −1. Each C57BL/6 mouse (H-2^{b}) or NOD mouse (H-2^{g7}) was infused with 4×10⁷ Balb/c bone marrow cells at day 0. Rapamycin (Rapa) was administrated by gavage at the dose of 2 mg/kg from day 0 to day 2, then 1 mg/kg once very two days until day 14. Anti-CD40L (MR1, 0.5 mg) was given i.p. at day 0 to day 5, then at day 7, 10 and 14. The level of donor-specific chimerism in peripheral blood was determined at different time points by flow cytometric analysis. Total number of chimeric mice and percentage of donor chimerism are shown as follows:

Induction of Mixed Chimerism in Balb/c to C57BL Strain Combination

| | Donor | | | |
|---|---|---|---|---|
| Conditioning | Cell | Immune | Mixed Chimerism | |
| Therapy | Treatment | Blockade | 4 Weeks | 8 Weeks |
| FL + CY | No | Rapa | 4/5, 7.7 ± 1.0% | 4/5, |
| | | | | 10.3 ± 1.9% |
| FL + CY | No | MR1 | 6/6, | 5/5, |
| | | | 34.5 ± 20.9% | 28.5 ± 10.3% |
| FL + CY | No | MR1 + Rapa | 5/5, 9.0 ± 7.4% | 4/5, 8.7 ± 4.6% |
| FL + CY | Yes | MR1 + Rapa | 6/6, | 6/6, |
| | | | 21.6 ± 4.3% | 24.9 ± 2.8% |
| FL | Yes | MR1 + Rapa | 0/6 | 0/6 |
| CY | Yes | MR1 + Rapa | 5/6, | 5/6, |
| | | | 11.5 ± 1.7% | 14.3 ± 2.7% |

Induction of Mixed Chimerism in Balb/C to NOD Strain Combination

| | Donor | | | |
|---|---|---|---|---|
| Conditioning | Cell | Immune | Mixed Chimerism | |
| Therapy | Treatment | Blockade | 4 Weeks | 8 Weeks |
| FL + CY | No | MR1 | 5/5, | 5/5, |
| | | | 81.6 ± 14.1% | 86.2 ± 16.2 |
| FL + CY | No | MR1 + Rapa | 6/6, | 6/6, |
| | | | 24.5 ± 10.0% | 26.1 ± 6.5% |
| FL + CY | Yes | MR1 + Rapa | 8/8, | 8/8, |
| | | | 56.3 ± 6.9% | 54.0 ± 15.1% |
| FL | Yes | MR1 + Rapa | 0/6 | 0/6 |
| CY | Yes | MR1 + Rapa | 6/6, | 5/5, |
| | | | 27.5 ± 1.7% | 17.3 ± 3.7% |

These studies demonstrated that high level of allogeneic mixed chimerism could be induced in C57BL/6 and NOD mice after transplantation of a modest bone marrow dose by using fludarabine and cyclophosphamide as conditioning therapy. Donor cell pretreatment enhances the induction of mixed chimerism.

### Example 2

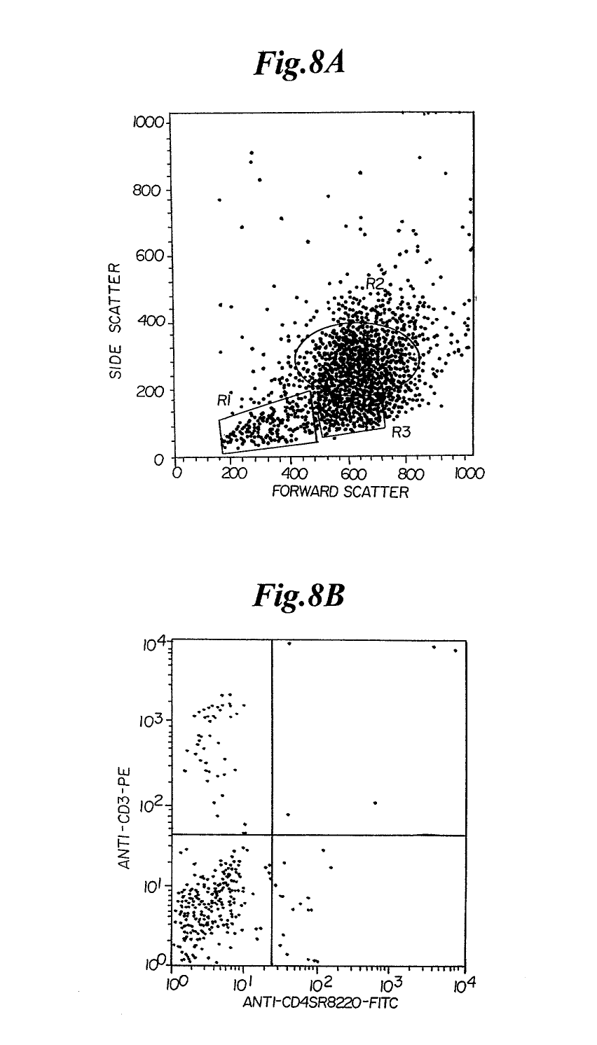

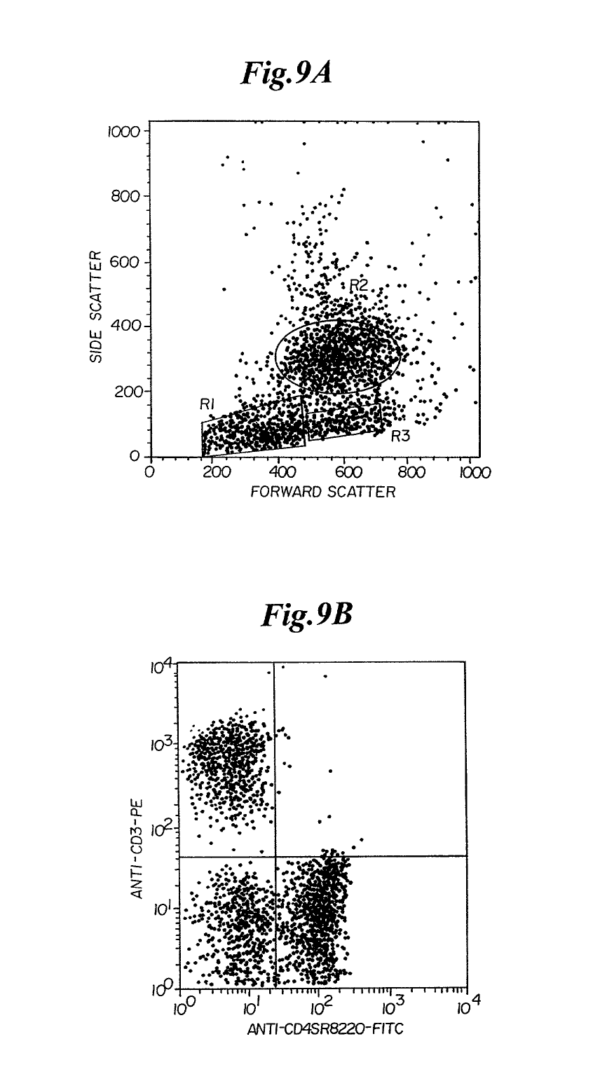

The conditioning therapy using FLU and CY was shown to avoid neutropenia. Five C57BL/6 mice were given FLU (400 mg/kg) and CY (200 mg/kg) as described in example 1 and five control mice received no treatment. After one week, blood samples were collecting and analyzed by flow cytometry using the CD3 marker for T cells and the CD45R/B220 marker for cells. Lymphocytes (R1) in the treated mice were depleted by FL and CY treatment (FIGS. 8a and 8b) compared with the control mice (FIGS. 9a and 9b). But granulocytes (R2) and monocytes (R3) were only slightly affected, showing that neutropenia was avoided.

### Example 3

These protocols for inducing mixed chimerism were found to cause the recipients to remove the donor-reactive T-cells from their blood. Balb/C mice express antigens that are attacked by V-Beta 5.5⁺ and V-Beta 11⁺ TCR bearing T-lymphocytes and therefore normal balb/C mice do not have V-Beta5.5⁺ and V-Beta11⁺ T-lymphocytes. Therefore when balb/C bone marrow is transplanted into other mouse strains, it is desirable that the recipient mice do not have lymphocytes that express V-Beta5.5⁺ and V-Beta11⁺. C57BL/6 mice, however, normally do have V-Beta5.5⁺ and V-Beta11⁺ lymphocytes. Therefore a mixed chimer that successfully integrates the immune systems of both Balb/C and C57BL/6 mice should not have V-Beta5.5⁺ and V-Beta11⁺ lymphocytes.

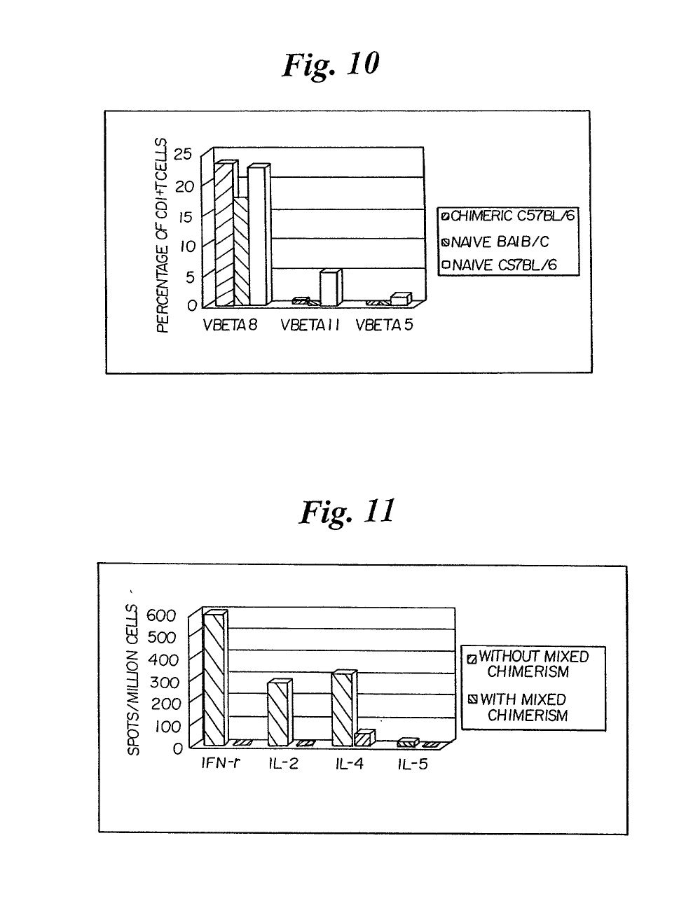

The protocols described herein were used to induce mixed chimerism was in C57BL/6 mice using Balb/c donor bone marrow FIG. 10). V-Beta usage of TCR was studied 20 weeks after bone marrow transplantation. These experiments showed that that V-Beta5.5+ and V-Beta11⁺ lymphocytes were almost completely eliminated in theses chimeric mice at 20 weeks after bone marrow (as shown by measurements of CD4⁺ lymphocytes). Control lymphocytes were lymphocyte levels were unchanged (measured V-Beta 8⁺ CD4⁺ T-cells). These experiments show that these methods for inducing mixed chimerism result in deletion of donor-reactive T-cells.

### Example 4

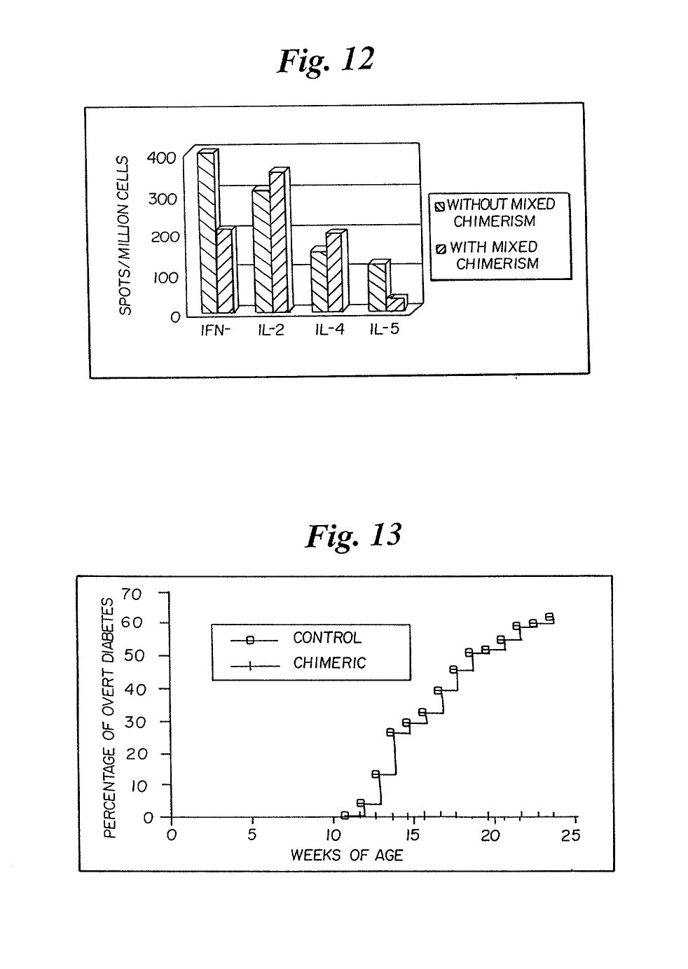

The donor immune system T-cells of the mixed chimers developed by the procedures described herein did not attack the host. The frequency of donor specific cytokine (interferon-gamma, IL-2, IL-4, and IL-5) producing T-cells in mixed chimeric NOD mice was measured by enzyme-linked immunospot assay (ELISPOT) assay a 20 weeks after bone marrow cell transplantation. Spleen cells from recipient chimeric mice and recipient non-chimeric mice were collected and cultured with donor cells or phytohemagglutinin (PHA) for 24 hours. Few donor specific cytokine producing T cells could be found in chimeric NOD mice compared to NOD mice without chimerism (FIG. 11). PHA mitogen specific cytokine secreting T cells were seen in both chimeric and non-chimeric NOD mice (FIG. 12).

### Example 5

The onset of diabetes in prediabetic mice was prevented by establishing mixed chimerism using the procedures described herein. NOD prediabetic mice were treated with conditioning treatment, bone marrow cell transplants, and immune blockade at 8-9 weeks of age and compared to untreated prediabetic NOD mice. Blood glucose levels were monitored (FIG. 13). At age 24 weeks, none of the 27 chimeric mice had developed diabetes but 61 of 100 of the control mice had developed diabetes. (p<0.01).

### Example 6

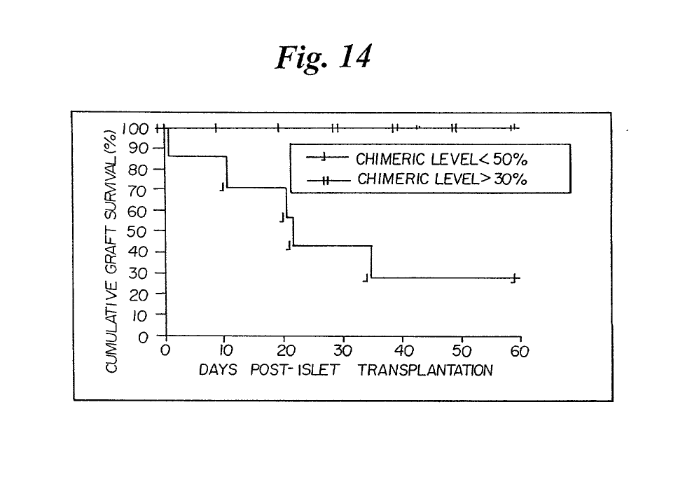

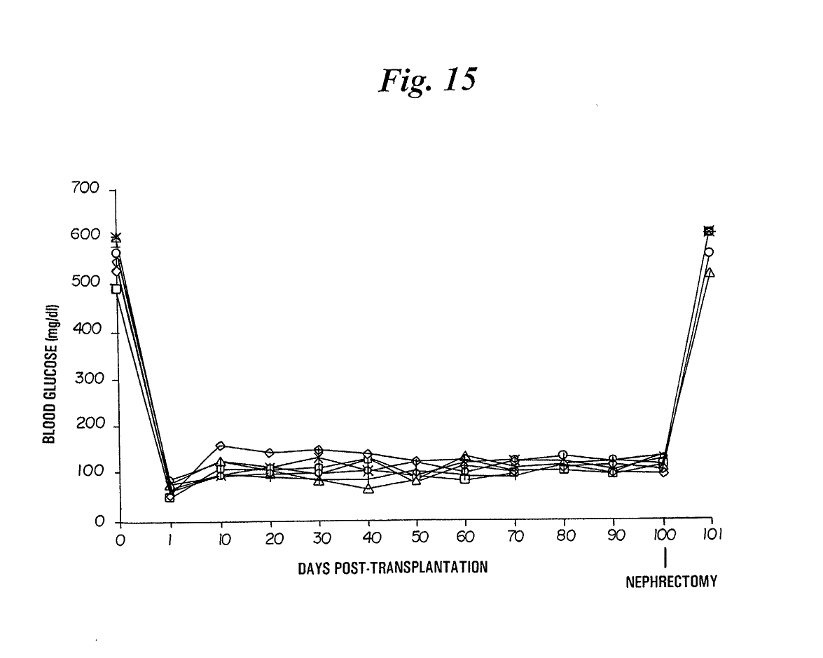

Diabetes was cured by inducing mixed chimerism in combination with a pancreatic islet transplant. NOD mice that had been diabetic for at least two weeks were given a donor-cell pretreatment of Balb/c spleen cells (1×10⁸) at d-3. FL (400 mg/kg) and CY (200 mg/kg) were given intraperitoneally on d-1. Balb/c bone marrow cells (4×10⁷) were given on do. Rapamycin was administered by gavage (2 mg/kg/day) from d0 to d2 and then every other day at 1 mg/kg/day until d14. Anti-CD154 (MR1, 0.5 mg) was given intraperitoneally daily from d0 to d5, then on d7, d10, and d14. Flow cytometry was used to measure donor-specific chimerism two weeks after bone marrow cell transplant. All pancreatic islet grafts survived over 60 days in chimeric mice with mixed chimerism levels of at least 30% donor cells at two weeks (FIG. 14). Islet grafts were rejected in 5 of 7 chimeric mice with less than 30% donor chimerism.

### Example 7

Diabetes was cured by simultaneous bone marrow cell and pancreatic islets. Preconditioning treatments of FL (200 mg/kg) and CY (100 mg/kg) were administered intraperitoneally to female recipient NOD mice at d-2 and d-1. Anti-lymphocyte serum (ALS, 0.3 ml) was given on d-1 and on d0. Four hundred MHC-matched male NOR islets were transplanted into the left kidney capsule of each diabetic female NOD mouse, and 1×10⁸ male NOR bone marrow cells were simultaneously injected intravenously. Rapamycin was administered at 1/mg/kg from d0 to d2 and then very other day until d14. NOR islet survival without any treatment was 8.0±2 days. FL and CY treatment prolonged islet graft survival to 23.5±8.5 days (p<0.05). ALS and rapamycin treatment and NOR bone marrow cell infusion also significantly prolonged NOR islet graft survival to 32.±2.5 days (p<0.01). However, all NOR islet grafts that survived over 100 days had simultaneous bone marrow cell/islet transplant and received Fl, CY, ALS, and rapamycin (Table Ex7-1). The return of hypoglycemia after nephrectomy confirmed that the islet grafts were functioning.

To further test whether donor-specific tolerance had been induced, donor NOR islets or third-party Balb/c islets were transplanted into the right kidney capsule of these mice. Donor-specific NOR islet grafts survived over 80 days and third-party Balb/c islet grafts were rejected in two weeks (Table Ex7-2). Donor-specific chimerism of peripheral blood in these mice was measured by semi-quantitative PCR for a male specific marker (SRY). The average percentage of this male NOR marker in DNA derived from peripheral blood of these female NOD mice at 100 days post-transplantation was 10%.

TABLE EX7-1

| Bone marrow | Conditioning | Islet Graft |
|---|---|---|
| cell Transplant | Treatment | Survival (Days × n) |
| No | None | 5, 6 × 2, 7 × 2, |
| | | 8 × 3, 9, 11 × 2, 12 |
| No | FL + Cy | 17, 23, 24, 40 |
| Yes | ALS + Rapamycin | 28, 32 × 2, 35 |
| Yes | FL + CY + ALS + Rapamycin | >100 × 7 |

TABLE EX7-2
Second Islet Graft Survival In Diabetic NOD Mice

| Donor | Treatment | Islet Graft Survival (Days*) |
|---|---|---|
| Balb/c | None | 12, 14 |
| NOR | None | >100 × 4 |
| | | >60 × 3 |

*the symbol “>” indicates that the mice are still alive and have not rejected their graft at the time of writing

### Example 8

This example shows methods and systems for inducing mixed hematopoietic chimerism without irradiation in a fully MHC-mismatched allogeneic bone marrow transplantation. This example shows that stable and high levels of mixed chimerism can be induced by irradiation-free nonmyeloablative approaches after transplantation of regular does of bone marrow in a fully MHC-mismatched mouse combination. Donor-specific transfusion (DST, 0.25 ml) was given a day −7. ALS (0.3 ml) was administered at day −8 and day −5. Busulfan (Bu, 20 mg/kg) and cyclophosphamide (Cy, 100 mg/kg) was given at day −3 and day −2. Bone marrow at a dose of 4×10⁷ from Balb/c mice were injected into each C57BL/6 mice at day 0. Anti-CD40L (MR1, 0.5 mg) was give at day 0, 2 and CTLA4Ig was given at day 2. Rapamycin (Rapa) was administrated at the dose of 2 mg/kg from day −1 and day 2, then 1 mg/kg once very two days until day 14. The level of donor-specific chimerism was determined at different time points by flow cytometry. The results of different groups were as follows:

TABLE EX8-1
Balb/c Donor Chimerism in PBL of C57BL/6 Mice at 8 Weeks
Post-Transplant

| | Condition- | | | | Percentage of |
|---|---|---|---|---|---|
| | ing | | Immune | Chimeric | Donor Cells |
| Group | Therapy | DST | Blockade | Mice | in Chimeric Mice |
| 1 | Bu + CY, | No | MR1 + | 5/5 | 34.3 ± 7.4% |
| | ALS | | CTLA4Ig + | | |
| | | | Rapa | | |
| 2 | Bu + CY, | Yes | MR1 + | 5/5 | 74.8 ± 4.8% |
| | ALS | | CTLA4Ig + | | |
| | | | Rapa | | |
| 3 | ALS | Yes | MR1 + | 0/6 | 0% |
| | | | CTLA4Ig + | | |
| | | | Rapa | | |
| 4 | Bu + CY | Yes | MR1 + | 1/6 | 38.9% |
| | | | CTLA4Ig + | | |
| | | | Rapa | | |
| 5 | Bu + CY, | Yes | Rapa | 6/6 | 76.8 ± 13.6% |
| | ALS | | | | |
| 6 | Bu + CY, | Yes | MR1 + | 4/5 | 63.7 ± 7.0% |
| | ALS | | CTLA4Ig | | |
| 7 | Bu + CY, | Yes | MR1 | 4/6 | 25.3 ± 3.3% |
| | ALS | | | | |
| 8 | Bu + CY, | Yes | CTLA4Ig | 3/6 | 18.2 ± 12.9% |
| | ALS | | | | |
| 9 | Bu + CY, | Yes | MR1 + Rapa | 6/6 | 50.3 ± 4.0% |
| | ALS | | | | |

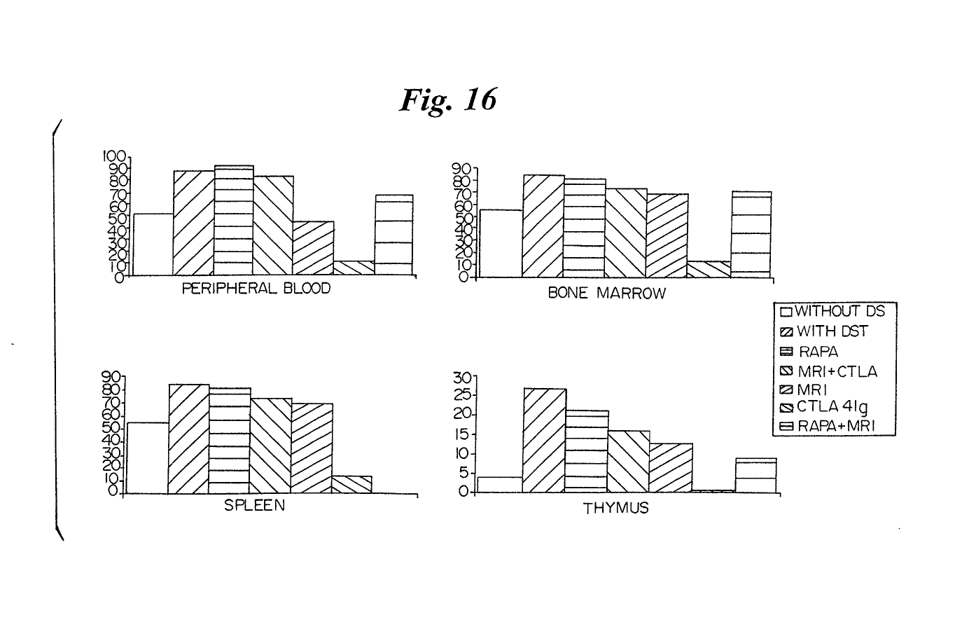

FIG. 16 shows the donor chimerism levels at 20 weeks in various hematopoietic organs.

These studies demonstrated that stable and high level of mixed chimerism could be induced in a fully MHC-mismatched mouse combination after transplantation of regular dose of bone marrow without any irradiation. Bu+Cy and ALS as conditioning therapy successfully induced mixed chimerism. Costimulatory blockades and Rapamycin alone or combination as post-bone marrow treatment helped to induce mixed chimerism. This approach may be used to induce donor-specific tolerance in clinical islet transplantation and living donor related solid organ transplantation.

### Example 9

### Induction Of Transient Chimerism in Nonhuman Primates Using Mildly Myeloablative Preconditioning Treatment and Immune Blockade

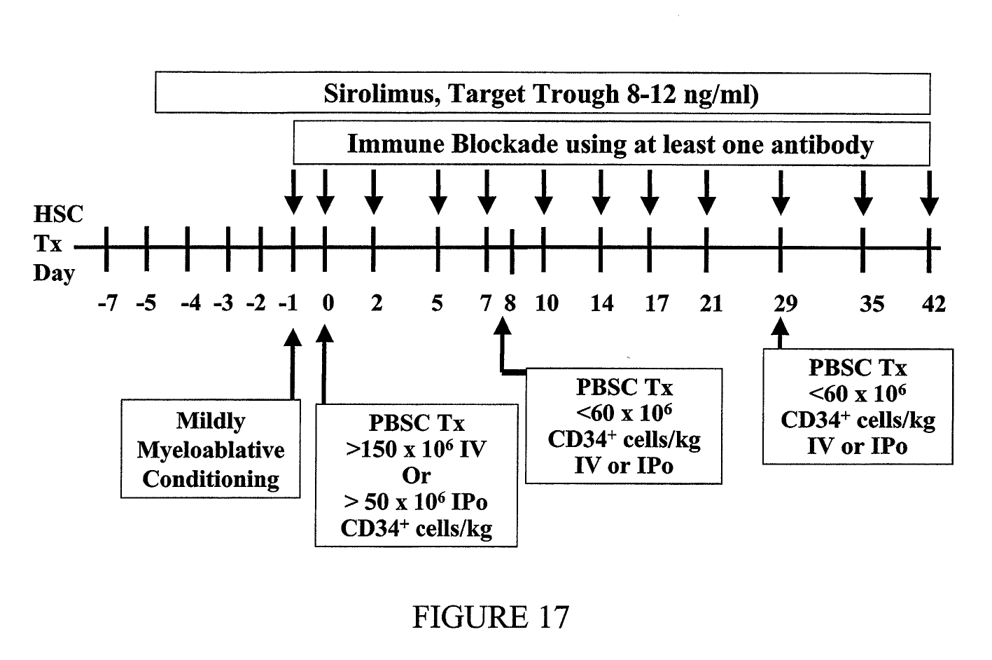

This example demonstrates certain embodiments of the invention for inducing transient chimerism in nonhuman primates. Despite the fact that certain components of the protocol have previously been employed by other investigators, no other reports are known that describe comparable levels and comparable durations of mixed hematopoietic chimerism in the preclinical, mismatched non human primate model. The methods of this Example are summarized in FIG. 17. The monkey was myelosuppressed with a preconditioning treatment of low dose total body irradiation (200 cGy). Prevention of rejection and GVHD was accomplished with an immune blockade treatment of sirolimus and anti-CD40L monoclonal antibody immunotherapy. Three peripheral blood stem cell (PBSC) transplantations (Tx) were performed on days 0, 8, and 29, respectively. PBSCs were collected from haplotype-mismatched donor animals.

The results are shown in FIG. 18, which is an electropherogram generated from capillary electrophoresis. The electropherograms represent the amplification of STR marker D11S925. The recipient lacked this marker pretransplant, FIG. 18A, but the marker was present in the donor FIG. 18B. Posttransplant, the recipient carried this marker in monocytes, FIG. 18C, and neutrophils, FIG. 18D. These results show the successful induction of mixed hematopoietic chimerism induction in a rhesus monkey and that PBSC transplantation for the induction of mixed chimerism was successful across a fully haplotype mismatched MHC barrier. Detection of chimerism and detailed materials, methods, and protocols are as per Example 10, below, unless otherwise stated.

### Example 10

### Induction Of Transient Mixed Chimerism and Tolerance Of Major Organs

This Example sets forth systems and methods for inducing transient mixed chimerism in humans and nonhuman animal recipients and demonstrates how the recipients are thereby made to tolerate transplantation of major organs from the donor. This Example is presented in terms of a procedure performed on nonhuman primates (NHPs) with pancreatic islet cells used as an example of a major organ system. Persons of ordinary skill in these arts, however, will immediately recognize how to perform this protocol for humans with pancreatic islet organs or other organ or tissue transplants.

Peripheral blood stem cell (PBSC) mobilization and collection procedures were performed in non human primates following protocols developed by Donahue et al.(39) To mobilize peripheral blood stem cells, granulocyte colony-stimulating factor (G-CSF) (100 mcg/kg/d) and stem cell factor (SCF) (400 mcg/kg/d) are administered subcutaneously for five days before collection. A FENWAL CS 3000-plus apheresis instrument peripherally mobilized hematopoietic stem cells. Blood is drawn from a femoral vein catheter and returned to an upper extremity peripheral venous catheter. General anesthesia is achieved with ketamine and 1% isoflurane. About 6 to 10 estimated blood volumes are processed in order to obtain an average of 4×10⁹ total nucleated cells containing >20×10⁶CD34+ cells in 13 of the last 15 leukapheresis products. These data indicate that techniques have been established that allow the transplantation of the target dose of >10×10⁶ CD34+ cells/kg recipient body weight. Further, islets were successfully transplanted from the PBSC donor to the recipient without rejection.

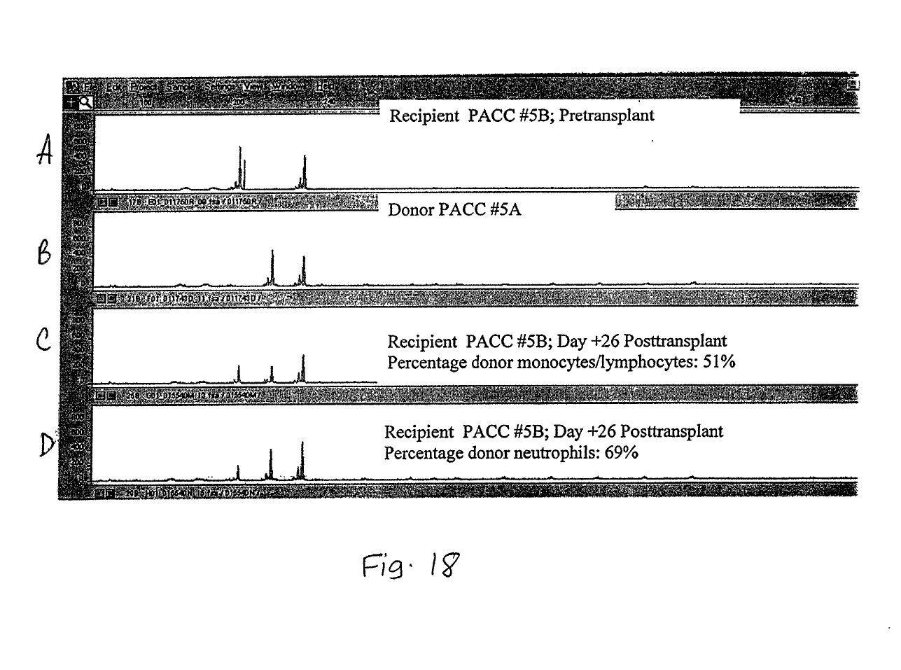

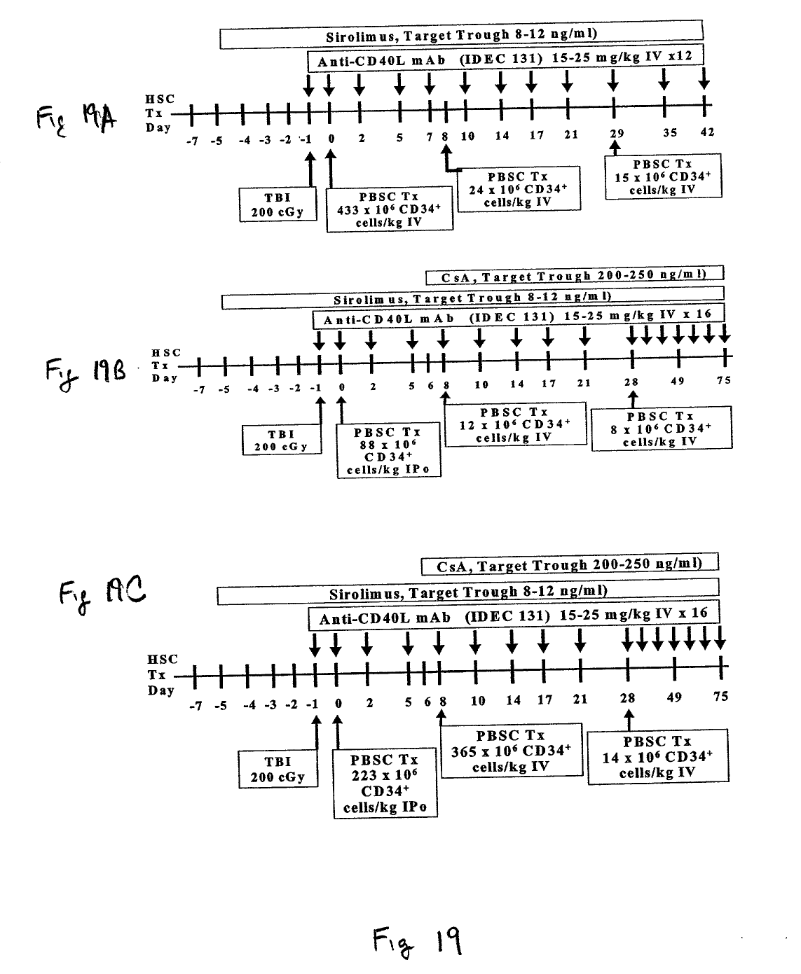

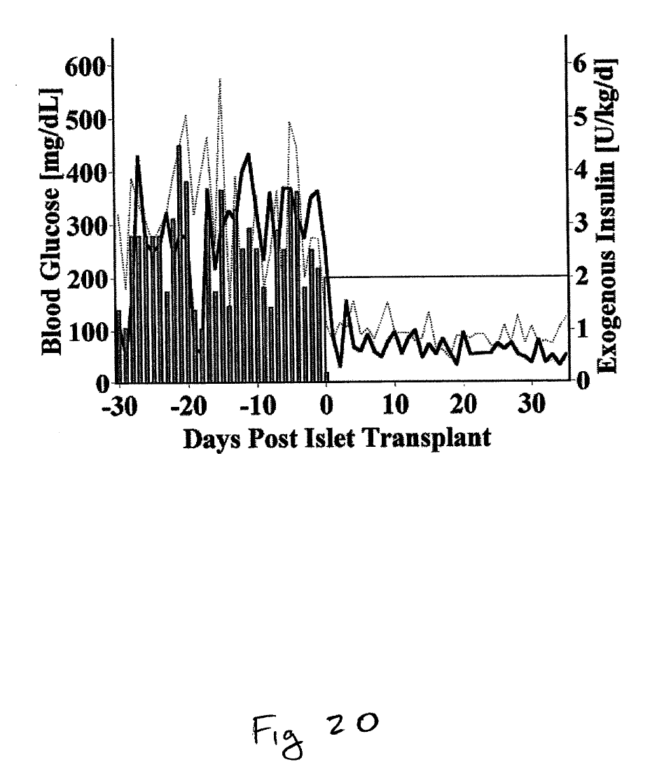

Table Ex10 below summarizes the mixed chimerism protocol and results in five consecutive recipient NHPs. Myelosuppression was limited to a single, 200-cGy dose of total body irradiation (TBI) administered on day −1 relative to the first infusion of mobilized PBSCs. Immunosuppression was with immune blockade using anti-CD40L mAB IDEC-131 (12 IV infusions of 15-25 mg/kg from days −1 to +42 in Group A and 16 infusions from days −1 to +75 in Group B), sirolimus (SRL; target trough 8-12 ng/ml; from day −5 to +42 in Group A, and from day −5 to +75 in Group B), and cyclosporine (CsA; target trough 200-250 ng/ml) was administered only to Group B animals. FIGS. 19A, 19B, and 19C show the treatment for animals 0IDP10, 011P06, and 011P01, respectively. Mobilized PBSC obtained from one-haplotype mismatched parental donors were infused intravenously (IV) in Group A and intraportally (IPo) in Group B as detailed in the table below.

The myelosuppressive and immunosuppressive therapy was well tolerated, the absolute neutrophil and platelet counts returned to normal levels by day 26 posttransplant, transfusion support was not required, and there was no clinical or laboratory evidence of acute GVHD. A profound neutropenia, i.e., less than 0.1×10⁹ cells/L of blood, was never observed. High levels of donor WBC chimerism were achieved in 1 of 3 Group A and in 2 of 2 Group B animals. Chimerism was transient and predominantly present in the myeloid lineages (sorting of lymphocytes prior to chimerism analysis revealed low-level or absent donor T lymphocyte chimerism). Representative output of one distinct STR genetic marker applied to the recipient sample and the donor DNA sample. The electropherograms represent the amplification of STR marker D11S925 analyzed by capillary electrophoresis on a 3100 GENETIC ANALYZER. The numbers above the peaks are the size of the PCR products in base pairs.

TABLE EX10

| | | | Immunosuppression | | | PBSC Infusion | | | % Donor WBC Chimerism | |
|---|---|---|---|---|---|---|---|---|---|---|
| | NHP | Myelo- | Anti- | | | (CD34⁺ cells, 10\ddot{ }6/kg) | | | (Day 28-Day 42-Day 63) | |
| Group | ID # | suppr. | CD40L | SRL | CsA | Day 0 | Day 8 | Day 28 | Neutrophils | Mono/Lympho |
| A | 01DP08 | TBI | X | X | — | 75.2 IV | 3.8 IV | 16.8 IV | 0-0-0 | 0-0-0 |
| A | 01DP10 | TBI | X | X | — | 433 IV | 24.0 IV | 14.8 IV | 69-57-5 | 51-22-3 |
| | (Fig 19A) | | | | | | | | | |
| A | 01IP08 | TBI | X | X | — | 22.7 IV | 4.2 IV | 13.5 IV | 0-0-0 | 0-0-0 |
| B | 01IP06 | TBI | X | X | X | 88.0 | 12.1 IV | 8.2 IV | 63-60-8 | 39-16-2 |
| | (Fig 19B) | | | | | IPo | | | | |
| B | 01IP01 | TBI | X | X | X | 223 IPo | 365.0 | 14.0 IV | 69-ND- | 50-ND- |
| | (Fig 19C) | | | | | | IV | | Pending | Pending |

To test for donor-specific immunologic tolerance, the survival of same-donor islet transplants was studied in NHPs 01DP10 and 011P08. Diabetes was induced on day +96 relative to the first PBSC infusion by IV injection of streptozotocin, and after confirmation of diabetes, an intraportal islet allograft from the previous parental PBSC donor was performed on day +126 (i.e., 84 days after discontinuation of immunosuppression). NHPs #01DP10 and #011P08 promptly achieved normoglycemia and insulin independence after islet transplantation (for the early post islet transplant course in NHP #01DP10, see FIG. 20; the bars show insulin requirements, the lines show am and pm blood glucose levels). The islet transplant continues to function in the previously chimeric NHP #01DP10 (greater than 104 days). NHP #01P08, which never achieved mixed chimerism after PBSC infusion, rejected the islet allograft on day +15 post islet transplant.

This Example shows that a major organ may be transplanted at least as late as four months after induction of transient chimerism and that the organ continues to be tolerated after the chimerism has dissipated. The recipients are typically more highly chimeric at earlier time points than later time points so an organ transplanted at earlier time points will be more easily accomplished. Thus it is possible to use the protocols set forth herein to perform transplants of bone marrow/stem cells within less than a week, preferably within less than three days, and more preferably within twenty four hours of the organ transplant. Such timing is particularly advantageous when the organs are recovered from a cadaver donor and the organs are not preserved, e.g., cryopreservation of pancreatic islets.

An advantage of the particular protocol used in this Example is the conditioning steps may be administered to the patient within less than seven days, preferably in less than three days, and more preferably within twenty four hours of a transplant. The recipient, however, is preferentially preconditioned with sirolimus for about five days prior to receiving a transplant from the donor.

The Protocols Below Were Followed for Examples 9-13 Except as Indicated Otherwise:

Peripheral blood stem cell mobilization, collection, and infusion: The technique for peripheral blood stem cell mobilization and collection in NHP described herein has been adapted from the protocols previously published by Donahue et al.(39) To mobilize peripheral blood stem cells, granulocyte colony stimulating factor (G-CSF) (100 mcg/kg/d) and stem cell factor (SCF) (400 mcg/kg/d) are administered subcutaneously for five days prior to collection. A Fenwal CS 3000 plus apheresis instrument (Baxter Healthcare Corp, Fenwal Division, Deerfield Ill.), dedicated for NHP use, is used to collect peripherally mobilized hematopoietic stem cells. The stem cell collection is performed by trained apheresis personnel under the direct supervision of a physician trained in apheresis. The apheresis instrument is primed with 120 mL of leukoreduced and irradiated rhesus whole blood and 120 mL of 5% human albumin. Heparin (100 U/kg) is administered i.v. and the stem cell collection is started at a rate of 5 to 10 mL/min; additional heparin (50 U/kg) is administered i.v. every hour during the procedure. Blood is drawn from a percutaneously placed central femoral vein catheter and returned to an upper extremity peripheral venous catheter. General anesthesia is achieved using ketamine and 1% isoflurane. The collection rate is increased to 20 to 30 mL/min as tolerated by the animal. The cell collection pump settings are programmed based on the donor's Hct and the Hct of the rhesus blood used to prime the instrument. Samples are taken for venous blood gases, CBC, PTT, and serum chemistries every 60 to 90 minutes during the procedure; K+ and Ca2+ are replaced as needed. Four to eight estimated blood volumes are processed in order to obtain an average of 4×109 total nucleated cells. If there are no planned collections within 21 days or if during the procedure the donor Hct falls below 22%, the extracorporal blood is reinfused to the donor. If the Hct is >22% and if there is a planned collection within 21 days the extra-corporeal blood may be transferred into a standard blood bag and stored for future use. ACD solution is added to the residual blood for preservation using the ratio of Blood:ACD 7:1. 5 mL of ACD is added to the stem cell product, which is weighed and sampled. Samples of the stem cell product are sent for total MNC counts, viability counts, and CD34+ cell counts. The product is maintained at room temperature and transfused to the recipient within 30 minutes. After the completion of the donor stem cell collection, the donor central access catheter is removed and direct pressure is held at the site. Protamine 0.5 mg/kg is administered over a five-minute period if bleeding from the central catheter-site persists. The peripheral i.v. is removed; the animal is allowed to awaken and is returned to an actively warmed cage. Close observation of the donor animal is maintained for 4 to 6 hours.

Monitoring for chimerism: Hematopoietic stem cell transplants, including bone marrow transplant, recipients are monitored for multilineage chimerism at 28, 42, 84, 200 and 365 days post stem cell transplant. To separate monocytes, lymphocytes and granulocytes, whole blood is stained using anti-human antibodies for CD14-FITC, CD3-PE, CD20-PE, and CD45-CyChrome (PHARMINGEN, San Diego, Calif.). Sorted populations are then analyzed for chimerism by PCR based amplification of a series of short tandem repeat DNA markers (STR's) followed by automated fluorescent analysis using the Model 373 DNA analyzer (APPLIED BIOSYSTEMS, Inc., Foster City, Calif.) combined with the GENESCAN™ software. Briefly, monitoring of genetic chimerism is performed by means of a series of highly polymorphic minisatellite and microsatellite markers amplified by PCR to construct the informative allelotypes for each of the donor and recipient animals prior to transplantation. The series of markers employ fluorochrome labeled oligonucleotide primers, and the PCR reactions are optimized to be analyzed in a multiplexed format on 12% denaturing polyacrylamide gels. The resulting distribution of allele specific bands are analyzed by sizing software that resolves the maternal and paternal alleles to within 2 base pairs. The series of markers selected provide is very high degree of probability of finding at least one unique donor and recipient specific allele that can be subsequently monitored when post transplant samples are analyzed. The percentage engraftment of the chimeric animal is calculated based on the quotient of the fluorescent signal of the donor alleles to the total alleles observed; and expressed as Percent Engraftment=(D1+D2)/(R1+R2+D1+D2), where D1 and D2 are the donor specific marker values and R1 and R2 are the recipient specific marker values.

### Example 11

### Protocols for Inducing Tolerance by Creating Mixed Chimerism

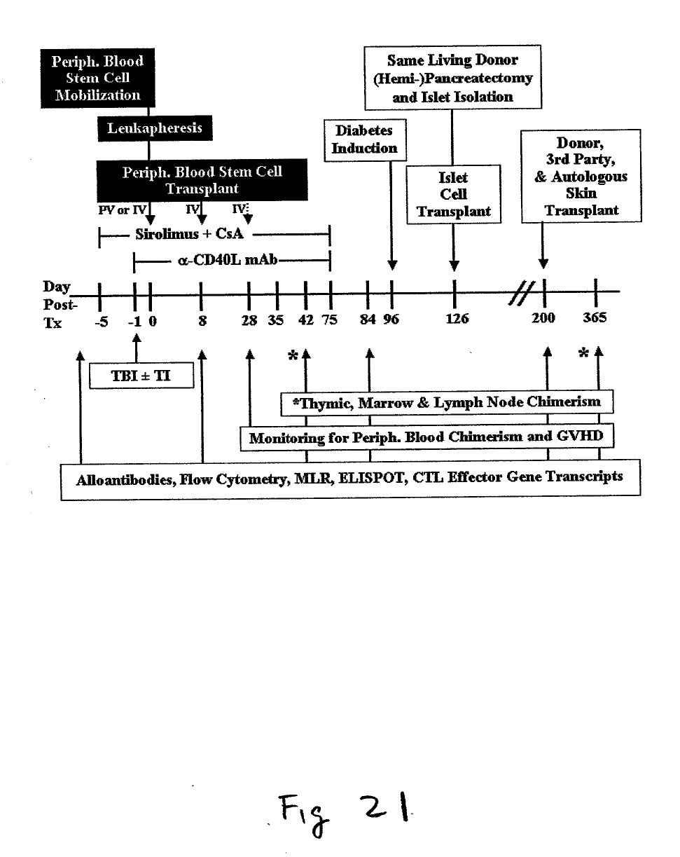

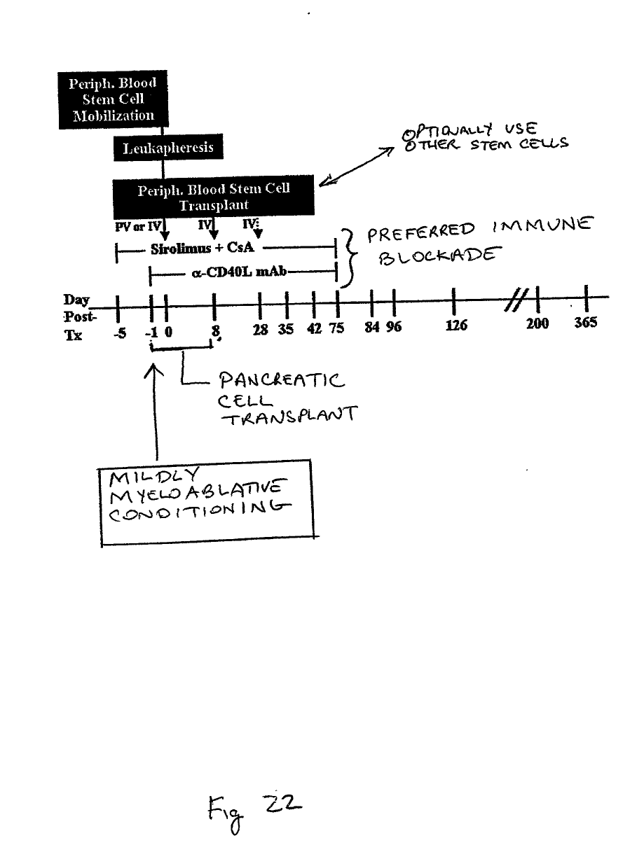

FIG. 21 depicts an embodiment of the invention for inducing mixed chimerism in combination with major organ transplants. In NHPs, diabetes induction is performed after the stem cell infusion. In humans, however, the pancreatic islet transplant is preferably performed within about seven days of the first infusion of stem cells, preferably within about three days, more preferably within about 24 hours and most preferably approximately simultaneously when the organs are from a human cadaver and the pancreatic islets are not frozen, e.g., by cryopreservation, see FIG. 22. TBI may be substituted with a course of mildly myeloablative treatments as described elsewhere in this Application. This Example is presented chiefly in terms of a NHP primate experiment with pancreatic islets but persons of ordinary skill in these arts will immediately apprehend use of the protocols for humans for pancreatic islets and other tissue and/or organ systems.

In this Example, NHPs will be transplanted following the previously developed mixed chimerism protocol (see Example 10, Group B in Table Ex10). Briefly, donor peripheral blood stem cell mobilization and collection will be performed in parent NHPs. Then unseparated leukapheresis products containing CD34+ stem cells will be infused intraportally as tolerizing antigen into haploidentical offspring on day 0 under the cover of anti-CD40L mAb and sirolimus. Sirolimus will be given from day −5 through day +75. The anti-CD40L mAb IDEC-131 will be administered IV 16 times from day −1 through day +75. CsA will not be introduced until day +5 and will be continued through day +75. PBSCs will again be mobilized and collected before intravenous infusion on days 8 and 28. One set of NHPs will further receive thymic irradiation (TI) on day −1 at a dose of 800 cGy to promote donor T cell chimerism and to facilitate stable mixed chimerism.

The CD34+ cell dose for all transplants will be >20×10⁶ cells/kg body weight. Anti-CD40L mAB (2) combined with sirolimus (11) and CsA (9) will be used for GVHD prophylaxis and engraftment augmentation through day 75 in all groups. NHPs will be monitored closely for multi-lineage chimerism, GVHD, and clinical and laboratory safety parameters. All NHPs will undergo thymic, marrow, and lymph node biopsies on days 42 and 365 after the first HCT.

To test for donor-specific immunologic tolerance, the functional survival of transplants and immune responses to primary and booster vaccinations will be analyzed. In all NHPs, regardless of the level of chimerism present at day 84 post hematopoietic stem cell transplantations, same-donor islet transplants will be performed on day 126, and same-donor and 3rd-party skin transplants on day 200 after the first PBSC transplants. Secondary outcome measures will be duration of normoglycemic and insulin-free islet transplant survival as well as skin transplant survival.

### Example 12

### Mixed Chimerism Protocols

This Example shows protocols that will be performed to induce stable mixed donor T-cell chimerism after intraportal, followed by intravenous, infusions of high-dose PBSCs in haploidentical, related NHP recipients and MHC filly mismatched NHPs given thymic irradiation, minimal myelosuppression, and temporary immunotherapy with anti-CD40L mAbs, sirolimus, and cyclosporine. This Example is presented in terms of NHPs but a person of ordinary skill in these arts will immediately apprehend how to apply this protocol to humans after reading this disclosure. Table Ex12 shows the protocols to be performed. TBI may be replaced by a course of mildly myeloablative preconditioning as described elsewhere in this Application.

TABLE EX12

| | | | | | | Donor PBMC/ | | |
|---|---|---|---|---|---|---|---|---|
| pro- | | Immunosuppression | | | | PBSC Infusion | | |
| tocol | Myelo- | Anti- | | | Thymic | IPo is intraportal; | | |
| # | suppr. | CD40L | SRL | CsA | Irradiation | IV is intravenous | | |
| 1 | TBI 200 | X | X | X | − | Day | Day | Day |
| | cGy | | | | | 0 | 8 | 28 |
| | | | | | | IPo | IV | IV |
| 2 | TBI 200 | X | X | X | X | Day | Day | Day |
| | cGy | | | | | 0 | 8 | 28 |
| | | | | | | IPo | IV | IV |

Protocol 1 will further demonstrate the safety and efficacy of the minimally myelosuppressive mixed chimerism protocol outlined in Example 10. Protocol 2 will use thymic irradiation to enhance these protocols. This protocol will be used to induce peripheral, and predominantly mycloid chimerism in one-haplotype mismatched NHPs. Note that these protocols avoid marked T-cell depletion and splenectomy, all of which have been previously found to be critical components of mixed chimerism strategies in NHPs.(18;23;44).

### Example 13

### Mixed Chimerism Protocols

This Example demonstrates protocols that will be performed to induce donor-specific immunologic tolerance and immunocompetence in NHPs with stable mixed hematopoietic chimerism. This Example is presented in terms of NHPs but a person of ordinary skill in these arts will immediately apprehend how to apply this protocol to humans after reading this disclosure. Irradiation may be replaced by a course of mildly myeloablative treatments as described elsewhere in this Application.

After conditioning therapy, initiation of anti-CD40L and sirolimus therapy, and stem cell transplants (preferably PBSC) on day 0, 8, and 28, recipient NHPs will be monitored for the presence of multilineage chimerism and GVHD. Anti-CD40L mAB, sirolimus, and CsA therapy will be discontinued on day 75. NHPs with and without stable donor T-cell chimerism at day 84 after their first PBSC transplant will receive streptozotocin i.v. on day 96 for diabetes induction, will undergo same-donor islet transplants on day 126, and will undergo autologous, same-donor and 3rd-party skin transplants on day 200.

Donor islets will be prepared from a living-donor segmental pancreas donation or a hemipancreatectomy specimen. The Applicants have established that a non chimeric recipient requires about 8,000-12,000 islet equivalents per kg body weight, but a chimeric recipient requires only about 2,000-4,000 islet equivalents per kg body weight. So a living donor may donate a portion of their pancreatic islets as well as stem cells/bone marrow to treat a patient for diabetes. Thus the Applicants will use fewer islets when transplanting islets into chimeric recipients than are conventionally used. The number of transplanted islets is preferably less than 6000, more preferably less than 3000, and yet more preferably 2000 or fewer islet equivalents per kg body weight. The islets are preferably transplanted from living donors but they may also be taken from human cadavers.

Advantages of using a living donor include that a the living donor may donate a portion of a pancreas and continue to have a functional pancreas. Further, the time available for establishing chimerism in the recipient is more flexible than when a cadaver donor is used because organ preservation is not a pressing issue. Moreover, repeated stem cell administrations over a time course are readily performed compared to a cadaver donor.

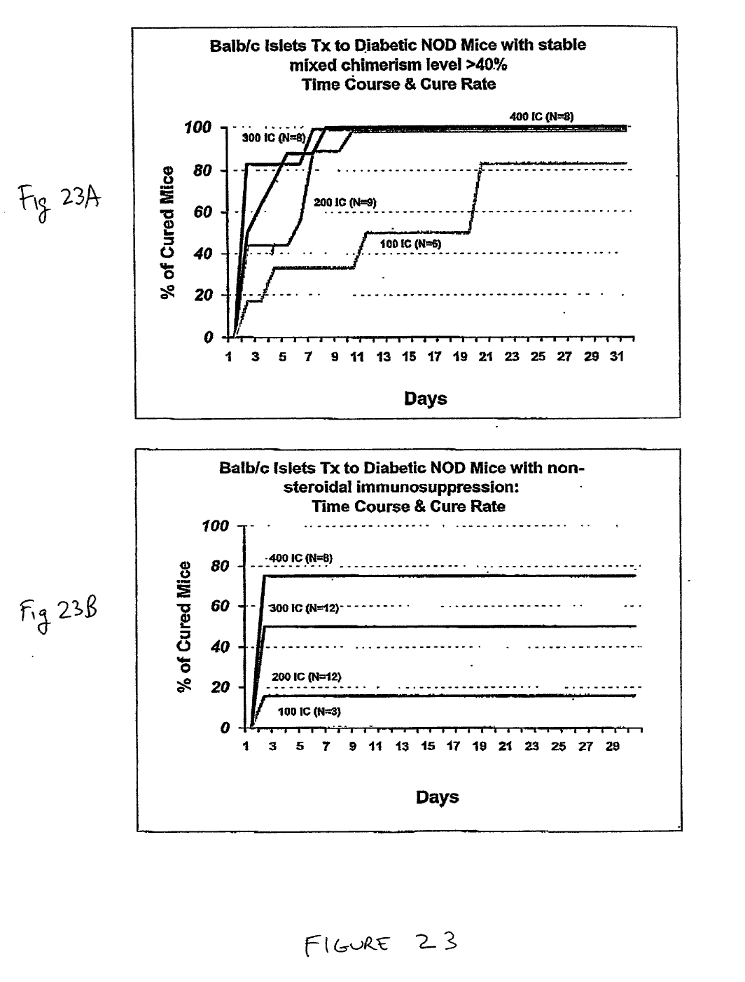

Referring to FIGS. 23A and 23B, the Applicants have already shown that fewer islets may be used to maintain insulin independence and normoglycemia in islet autograft recipients, who are not immunosuppressed, as compared to islet allograft recipients, who are immunosuppressed. Balb/c islets were transferred to diabetic NOD mice that had at least 40% mixed chimerism, which was stably induced using a conditioning treatment and immune blockade according to protocols already described herein. Transplant recipients received 100, 200, 300, or 400 islets (IC) at day 0. Chimeric mice (FIG. 23A) required fewer islets to be cured of diabetes compared to immunosuppressed mice (FIG. 23B). For example, 200 islets consistently cure all chimeric mice but cure only 15% of nonchimeric mice.

Immunocompetence studies will assess the immune responses to primary and booster vaccinations to tetanus toxoid done before, and hepatitis B vaccinations done after, preparative therapy, PBSC infusion, and anti-CD40L mAb, sirolimus and CsA therapy. In addition, cellular responses to mitogen and 3rd-party alloantigen will be monitored. Recipient NHPs will be sacrificed at day 365 after their first HCT. Islet allograft functional survival will be measured as a function of the level and duration of mixed hematopoietic chimerism.

### Example 14

### Mixed Chimerism Protocols with Sirolimus and Anti-CD40L

This Example shows embodiments of the invention that use an immune blockade of anti-CD40L monoclonal antibody (mAB) combined with sirolimus (RAPAMYCIN). These agents can either enhance or inhibit the induction of mixed chimerism depending on the preconditioning step.

In clinical bone marrow transplantation (BMT), post-BMT treatment is often used to treat graft-versus-host disease. The Applicants, without being limited to a particular theory of action, believe that post-BMT treatment can enhance bone marrow engraftment by suppressing the host-versus-graft response if pretransplant conditioning is less intensive but that it can inhibit bone marrow engraftment by suppressing the graft-versus-host response if conditioning is intensive. Thus the Applicants used a conditioning step that is mildly myeloablative so that it is less severe than many conventionally practiced treatments. This protocol is nonirradiative and the conditioning step may be used to replace TBI or partial TBI steps.

Balb/c mouse (H-2^{b}) splenocytes were injected into NOD mouse (H-2^{g7}) or C57BL/6 mouse (H-2^{d}) at day −3. Fludarabine phosphate (FL) and/or cyclophosphamide (CY) were given at day −1. Bone marrow cells (4×1 07) were transplanted at day 0. Anti-CD40L mAB (MR1) and sirolimus (RAPAMYCIN, Rapa) were given from day 0 to day 14. Donor-derived cells were measured by flow cytometric analysis at different time points. The proportion of mice with mixed chimerism and percentage of donor-derived cells in the chimeric mice at 4 weeks post-BMT in different groups are shown in Table Ex14.

TABLE EX14
Nonirradiative protocol for establishing mixed chimerism

| | Recipient | Conditioning | Posttransplant | 4 Weeks Post- |
|---|---|---|---|---|
| Group | Mice | Therapy | Treatment | BMT* |
| 1 | NOD | CY | Sirolimus | 0/7 |
| 2 | NOD | CY | MR1 | 5/7, 16.7 ± 11.5% |
| 3 | NOD | CY | MR1 + Sirolimus | 11/11, 29 ± 5.8% |
| 4 | NOD | FL | MR1 + Sirolimus | 0/6 |
| 5 | NOD | CY + FL | Sirolimus | 4/5, 95.4 ± 1.2% |
| 6 | NOD | CY + FL | MR1 | 6/6, 93.05 ± 7.9% |
| 7 | NOD | CY + FL | MR1 + Sirolimus | 7/8, 56.3 ± 6.9% |
| 8 | C57BL/6 | CY | Sirolimus | 2/6, 6.8 ± 6.1% |
| 9 | C57BL/6 | CY | MR1 | 12/12, 7.2 ± 4.4% |
| 10 | C57BL/6 | CY | MR1 + Sirolimus | 21/21, |
| | | | | 11.9 ± 9.2% |
| 11 | C57BL/6 | FL | MR1 + Sirolimus | 0/6 |
| 12 | C57BL/6 | CY + FL | Sirolimus | 4/4, 78.4 ± 21.1% |
| 13 | C57BL/6 | CY + FL | MR1 | 4/4, 86.8 ± 7.0% |
| 14 | C57BL/6 | CY + FL | MR1 + Sirolimus | 6/6, 21.6 ± 4.4% |

*number chimeric/total treated, % chimerism ± standard deviation

These data show that anti-CD40L mAB and sirolimus combination treatment may be used to significantly enhance the induction of mixed chimerism when CY alone was used as conditioning therapy.

### Example 15

### Protocols for Inducing Mixed Chimerism with FLU+CY Preconditioning and Immune Blockade With Anti-CD154

These protocols demonstrate methods and systems for inducing mixed chimerism with FLU+CY preconditioning and immune blockade with Anti-CD154 and an optional T-cell depletion step. T-cell-depleted allogeneic bone marrow transplantation may prevent GVHD but depleting T cells from allogeneic bone marrow often results in failure of bone marrow engraftment. T cells were depleted from bone marrow with anti-Thy-1.2 mAB and complement. Further preconditioning of fludarabine phosphate (FLU) and cyclophosphamide (CY) were given at day −1. Unmodified (4×10⁷) or T cell-depleted (2×10⁷) Balb/c mouse (H-2^{d}) bone marrow were transplanted into each NOD mouse (H-2g7) or C57BL/6 mouse (H-2^{b}). Immune blockade using anti-CD154 mAB was given i.p. at 0.5 mg from day 0 to day 5, then days 7, 10, and 14. Bone marrow engraftment was monitored by measuring donor MHC antigens through flow cytometric analysis at different time points. The proportion of mice with mixed chimerism and percentage of donor-derived cells in recipient mice at 2, 4 and 6 weeks posttransplant are shown in Table Ex 15.

TABLE EX15
Protocols for Inducing Mixed Chimerism with FLU + CY Preconditioning
and Immune Blockade With Anti-CD154

| | | | Anti- | | |
|---|---|---|---|---|---|
| | Pre- | | CD154 | | |
| Recipient | Condition | Bone Marrow | mAB | 4 Weeks | 6 Weeks |
| NOD | FLU + CY | Unmodified | No | 6/6, 97.1 ± 0.7% | 5/5, 96.1 ± 4.1% |
| NOD | FLU + CY | Unmodified | Yes | 5/5, 81.2 ± 14.2% | 5/5, 86.2 ± 12.2% |
| NOD | FLU + CY | T cell-depleted | No | 2/6, 2.3 ± 0.3% | 2/6, 7.9 ± 5.0% |
| NOD | FLU + CY | T cell-depleted | Yes | 6/6, 77.3 ± 11.8% | 6/6, 62.3 ± 24.5% |
| C57BL/6 | FLU + CY | T cell-depleted | No | 1/5, 22.9% | 1/5, 15.7% |
| C57BL/6 | FLU + CY | T cell-depleted | Yes | 4/5, 42.5 ± 15.3% | 4/5, 37.5 ± 14.7% |

Without anti-CD154 mAb immune blockade treatment, the percentage of recipient NOD's CD3⁺ T cells in peripheral lymphocytes was 38.6±8.8%, and the percentage of recipient C57BL/6's CD3⁺ T cells was 17.6±4.2% at 4 weeks post-T cell-depleted bone marrow transplantation. With anti-CD154 mAB immune blockade treatment, recipient CD3⁺ T cells were significantly decreased after T cell-depleted bone marrow transplantation. The percentage of recipient NOD's CD3⁺ T cells in peripheral lymphocytes was 10.3±4.0%, and the percentage of recipient C57BL/6's CD3⁺ T cells was 8.2±0.9% at 4 weeks. Donor Balb/c's CD3⁺ T cells were also detected in these anti-CD154 mAb treated recipients after T cell-depleted bone marrow transplantation. In recipient NOD mice, The percentage of donor Balb/c's CD3⁺ T cells in peripheral lymphocytes was 2.7±1.0% at 4 weeks, and 9.6±4.4% at 6 weeks. In recipient C57BL/6 mice, the percentage of donor Balb/c's CD3⁺ T cells in total lymphocytes was 2.2+0.8% at 4 weeks, and 6.3±3.8% at 6 weeks.

These results indicate that T cell-depleted bone marrow transplantation results in poor bone marrow engraftment in NOD mice and C57BL/6 mice using fludarabine phosphate and cyclophosphamide preconditioning combination as a mildly myeloablative and irradiation-free conditioning therapy. However, immune blockade of the CD40/CD154 pathway maybe used to enhance T cell-depleted bone marrow engraftment. Donor T cells facilitate bone marrow engraftment and immune blockade of the CD40/CD154 pathway replaces donor T cells to promote T cell-depleted stem cell survival and self-renewal. Thus a T-cell depletion step may be avoided. Alternatively, a T-cell depletion step may be used and the protocol performed using mildly myeloablative preconditioning and immune blockade.

### REFERENCES

References cited by number are provided below. These references, and all references cited in this Application, are hereby incorporated herein by reference.

1. McSweeney P A, Storb R. Biology of Blood and Marrow Transplantation 1999;5:192-203.

2. Blazar B R, Taylor P A, Panoskaltsis-Mortari A, Buhlman J, Xu J, Flavell R A et al. J Immunol 1997;158(1):29-39.

3. Wekerle T, Sayegh M H, Hill J, Zhao Y, Chandraker A, Swenson K G et al. J. Exp. Med. 1998;187(12):2037-44.

4. Wekerle T, Sayegh M H, Ito H, Hill J, Chandraker A, Pearson D A et al. Transplantation 1999;9:1348-55.

5. Taylor P A, Lees C J, Waldmann H, Noelle R J, Blazar B R. Blood 2001;98(2):467-74.

6. Durham M M, Bingaman A W, Adams A B, Ha J, Waitze S Y, Pearson T C et al. J. Immunol. 2000;165:1-4.

7. Quesenberry P J, Zhong S, Wang H, Stewart M. Blood 2001;97(2):557-64.

8. Adams A B, Durham M M, Kean L, Shirasugi N, Ha J, Williams M A et al. J. Immunol. 2001;167:1103-11.

9. Taylor P A, Lees C J, Noelle R J, Blazar B R. Abstract submitted to ASH 2001 meeting 2001.

10. Wu, Heuss, N., Levayyoung, B. K., et al. Transplantation (in press), 2002.

11. Blazar B R, Taylor P A, Sehgal S N, Vallera D A. Blood 1994;83(2):600-9.

12. Hale D A, Gottschalk R, Fukuzaki T, Wood M L, Maki T, Monaco A P. Transplantation 1997;63(3):359-64.

13. Wu, T., Sozen, H., Luo, B., et al. Bone Marrow Transplant (in press), 2002.

14. McSweeney P, Niederwieser D, Shizuru J, Sandmaier B M, Molina A J, Maloney D G et al. Blood 2001;97(11):3390-400.

15. Ildstad S T, Sachs D H. Nature 1984;307(5947):168.

16. Sharabi Y, Sachs D H. J Exp Med 1989;169:493.

17. Huang C A, Fuchimoto Y, Scheier-Dolberg R, Murphy M C, Neville D M J, Sachs D H. J Clin Invest 2000;105:173.

18. Kawai T, Cosimi B, Colvin R, Powelson J, Eason J, Kozlowski T et al. Transplantation 1995;59:256-62.

19. Wekerle T, Sayegh M H, Hill J, Zhao Y, Chandraker A, Swenson K G et al. J. Exp. Med. 1998;187(12):2037-44.

20. Wekerle T, Sykes M. Annu Rev Med 2001;52:353-70.

21. Wekerle T, Kurtz J, Sayegh M H, Ito H, Wells A, Bensinger S et al. J Immunol. 2001:2311-6.

22. Taylor P A, Noelle R J, Blazar B R. J Exp Med 2001; 193(11):1311-8.

23. Kawai T, Abrahamian G, Sogawa H, Wee S, Boskovic S, Andrew D et al. Transplant. Proc. 2001;33:221-2.

24. Wekerle T, Kurtz J, Ito.H., et al. Nature Medicine 2000;6:464.

25. Fuchimoto Y, Huang C A, Yamada K, Shimizu A, Kitamura H, Colvin R B et al. J. Clin. Invest. 2001;105(12): 1779-89.

26. Rachamim B, Nan J, Segall H, Krauthgamer R, Marcus H, Berrrebi A et al. Transplantation 1998;65:1386-93.

27. Reich-Zeliger S, Zhao Y, Krauthgamer R, Bachar-Lustig E, Reisner Y. Immunity. 2000;13:507-15.

28. Reisner Y, Martelli M F. Exp. Hematol. 2000;28:119-27.

29. Aversa F, Tabilio A, Velardi A, Cunningham I, Terenzi A, Falzetti F et al. N. Engl. J. Med. 1998;339(17):1186-93.

30. Chase W M. Proc. Soc. Exp. Biol. Med. 1946;61:257-9.

31. Morita H, Sugiura K, Inaba M, Jin T, Ishikawa J, Lian Z et al. Proc. Nat. Acad. Sci. U.S.A. 1999;95:6947-52.

32. Morita H, Nakamura N, Sugiura K, Satoi S, Sakakura Y, Tu W et al. Ann. Surg. 1999;230(1):114-9.

33. Kushida T, Inaba M, Ikebukuro K, Ngahama T, Oyaizu H, Lee S et al. (Dayt) 2000; 18(6):453-6.

34. Trivedi H L, Shah V R, Shah P R, Sane A S, Vanikar A V, Trivedi V B et al. Transplant Proceedings 2001;33:71-6.

35. Li Y, Li X C, Zheng X X, Wells A D, Turka L A, Strom T B. Nat. Med 1999;5(11):1298-302.

36. Sykes M, Szot G L, Swenson K G, Pearson D A. Nature Medicine 1997;3(7):783-7.

37. Auchincloss H. American Journal of Transplantation 2001;1:6-12.

38. Kirk A D, Harlan D M. Current Opinion in Transplantation 2000;5:108-13.

39. Donahue R E, Kirby M R, Metzger M E, Agricola B A, Sellers S E, Cullis H M. Blood 1996;87:1644-53.

40. Wekerle T, Sykes M. Transplantation 1999;66:459.

41. Han D, Xu X, Pastori R L, Ricordi C, Kenyon N S. Diabetes 2002;51:562-6.

42. Tomita, Y., Khan, A., and Sykes, M. J. Immunol 153, 1087-1098.1994.

43. Sykes M, Szot G L, Swenson K, Pearson D A, Wekerle T. Exp Hematol. 1998;26(6):457-65.

44. Kimikawa M, Sachs D H, Colvin R B, Bartholomew A, Kawai T, Cosimi A B. Transplantation 1997;64:709.

45. Kawai T, Poncelet A, Sachs D H, Mauiyyedi S, Boskovic S, Wee S L et al. Transplantation 1999;68:1767.

46. Storb R, Yu C, Zaucha J M, Deeg H J, Georges G, Kiem H P et al. Blood 1999;94(7): 2523.

47. Sugiura K, Kato K, Hashimoto F, Jin T, Amoh Y, Yamamoto Y et al. Immunobiol. 1997; 197:460-77.

48. Parker D C, Greiner D L, Phillips N E, Appel M C, Steele A W, Durie F H et al. Proc. Nat. Acad. Sci. U.S.A. 1995;92(21):9560-4.

49. Blazar B R, Taylor P A, Noelle R J, Vallera D A. J Clin Invest 1998;102(3):473-82.

50. Slavin S, Nagler A, Naparstek E, et al. Blood 1998;91:756-63.

51. Sutherland D E R, Najarian J S, Gruessner R W. Transplant. Proc. 1998;30:2264-6.

52. Wahoff D C, Papalois B E, Najarian J S, Kendall D M, Farney A C, Leone J P et al. Ann. Surg. 1995;222(4):562-75.

53. Dieckmann D, Plottner H, Berchtold S, Berger T, Schuler G. J Exp Med 2001;193(11):1303-10.

54. Nikolic B, Zhao G, Swenson K, Sykes M. Blood 2000;96(3):1166-72.

55. Nikolic B, Khan A, Sykes M. Biol Blood Marrow Transplant 2001;7(3):144-3.

56. Glick R D, Pearce I A, Trippett T, Saenz N C, Ginsberg R J, La Quaglia M P. J. Pediatr. Surg. 1999;34:559-64.

57. Heeger, P. S., Greenspan, N. S., Kuhlenschmidt, S. et al. J. Immunol. 163, 2267-2275. 1999.

58. Strehlau J, Pavlakis M, Lipman M, Shapiro M, Vasconcellos L, et al. Proc. Nat. Acad. Sci. U.S.A. 1997;94(2):695-700.

59. Vasconcellos L, Asher F, Schachter D, Zheng X X, Vasconcellos L H B, Shapiro M et al. Transplantation 1998;66(5):562-6.

60. Baogui L, Hartono C, Ding R, Sharma V K, Ramaswamy R, Qian B et al. N. Engl. J. Med. 2001;344:947-54.

61. Higuchi R, Dollinger G, Walsh P S, Griffith R. Biotechnology 1992;10(4):413-7.

62. Morrison T B, Weis J J, Wittwer C T. Biotechniques 1998;24(6):954-62.

63. Higuchi R, Fockler C, Dollinger G, Watson R. Biotechnology 1993;11(9):1026-30.

1. A method of treating a primate with materials from a donor, the method comprising the steps of:
transplanting donor cells from the donor into the primate that cause the production in the primate of immune system cells having at least one cellular marker that is characteristic of the donor immune system; and
creating a mixed chimeric immune system in the primate that is a chimer of the immune systems of the donor and the primate by a process that includes:
administering a mildly myeloablative conditioning treatment to the primate that avoids profound neutropenia in the primate; and
administering an immune blockade treatment to the primate;
such that the mixed chimeric immune system is at least 0.1% donor-specific as measured in peripheral blood of the primate.

2. The method of claim 1, wherein creating the mixed chimeric immune system is performed such that an absolute neutrophil count above at least 0.2×10⁹ cells per liter is maintained in the primate during the step of creating the mixed chimeric immune system.

3. The method of claim 1, wherein transplanting the donor cells is performed by administering bone marrow to the primate.

4. The method of claim 1, wherein transplanting the donor cells is performed by administering stem cells to the primate.

5. The method of claim 1, wherein transplanting the donor cells is performed by administering hematopoietic stem cells to the primate.

6. The method of claim 5, wherein transplanting the donor cells includes administering the stem cells to the primate on more than one day.

7. The method of claim 4, wherein the donor cells include stem cells collected from the peripheral blood of the donor.

8. The method of claim 4, wherein the donor cells include stem cells from a stem cell bank.

9. The method of claim 1 wherein administering a mildly myeloablative conditioning treatment includes administering a total body irradiation dose of less than 500 cGy.

10. The method of claim 1 wherein administering a mildly myeloablative conditioning treatment includes administering a total body irradiation dose of less than 300 cGy.

11. The method of claim 1, further comprising pretreating the primate with an infusion of donor antigen from the donor prior to transplantation of the donor cells.

12. The method of claim 1, wherein the conditioning treatment includes at least one drug chosen from the group consisting of fludarabine phosphate, busulfan, and cyclophosphamide.

13. The method of claim 1, wherein administering the conditioning treatment includes administering at least one drug chosen from the group consisting of a purine nucleoside analog, deoxycoformycin, 2-chloro-2′ deoxyadenosine, ifosamide, etoposide, mitoxantrone, doxorubicin, cisplatin, carboplatin, cytarabine, paclitaxel, nitrosoureas, melphalan, thiotepa, antilymphocyte serum, anti-thymocyte globulin, and anti-lymphocyte globulin.

14. The method of claim 1, wherein administering the immune blockade treatment is accomplished by administering immune blockade agents that interfere with costimulation.

15. The method of claim 14, wherein administering immune blockade agents that interfere with costimulation is accomplished by administering at least one of the compounds selected from the group consisting of anti-CD40L, anti-CD40, sirolimus, CTLA4Ig, LEA29Y, and compounds that inhibit the binding of B7 to CD28.

16. The method of claim 1, wherein the conditioning treatment is started less than five days prior to the transplantation of the donor cells.

17. The method of claim 1 wherein the transplantation of the donor cells includes a first infusion of the donor cells that is infused intraportally at a concentration of at least 50×10⁶ cells/kg of recipient.

18. A method of transplanting diabetes treating cells from a donor to a recipient, the method comprising:
administering diabetes treating cells from the donor to the recipient; and
inducing a state of mixed chimerism in the recipient with a process that includes:
infusing donor cells from the donor into the recipient, the donor cells causing the production of immune system cells in the recipient having at least one cellular marker that is characteristic of the donor immune system;
administering a conditioning treatment to the recipient that is mildly myeloablative; and
administering an immune blockade treatment to the recipient.

19. The method of claim 18, wherein a donor chimerism level of at least 5% is achieved by inducing the state of mixed chimerism as determined by measurements taken from at least one sample of the recipient's peripheral blood samples.

20. The method of claim 18, further comprising administering a cell pretreatment from the donor to the recipient prior to the infusion of the donor cells.

21. The method of claim 20, further comprising administering an antilymphocyte serum within 48 hours after an end of the cell pretreatment.

22. The method of claim 18, wherein infusing donor cells includes administering stem cells to the recipient.

23. The method of claim 22, wherein infusing donor cells includes administering stem cells to the recipient on more than one day.

24. The method of claim 22, wherein infusing donor cells includes infusing stem cells collected from a stem cell bank or the donor's peripheral blood.

25. The method of claim 18 wherein administering a mildly myeloablative conditioning treatment includes administering a total body irradiation dose of less than 500 cGy.

26. The method of claim 25 wherein administering a mildly myeloablative conditioning treatment includes administering a total body irradiation dose of less than 300 cGy.

27. The method of claim 25 wherein a donor chimerism level of at least 30% is achieved by inducing the sate of mixed chimerism as determined by measurements taken format least one sample of the recipient's peripheral blood samples.

28. The method of claim 18, wherein the conditioning treatment includes at least one drug chosen from the group consisting of fludarabine phosphate, busulfan, and cyclophosphamide.

29. The method of claim 18, wherein administering the immune blockade treatment is accomplished by administering immune blockade agents that interfere with costimulation.

30. The method of claim 29, wherein administering immune blockade agents that interfere with costimulation is accomplished by administering at least one of the compounds selected from the group consisting of anti-CD40L, anti-CD40, sirolimus, CTLA4Ig, LEA29Y, and compounds that inhibit the binding of B7 to CD28.

31. The method of claim 18, wherein inducing the state of mixed chimerism is performed such that an absolute neutrophil count in the recipient remains above at least 0.5×10⁹ cells per liter throughout the inducing of the state of mixed chimerism.

32. The method of claim 18 wherein the donor is a cadaver and the primate receives the donor cells within three days of receiving the diabetes treating cells.

33. A method of treating a primate with materials from a donor, the method comprising:
transplanting donor cells from the donor into the primate that cause the production of immune system cells in the primate having at least one cellular marker that is characteristic of the donor immune system; and
creating a mixed chimeric immune system in the primate that is a chimer of the immune systems of the donor and the primate by a method that includes:
administering a conditioning treatment to the primate that includes radiation; and
administering an immune blockade treatment to the primate;
such that the mixed chimeric immune system is at least 5% donor-specific as measurable in peripheral blood of the primate and an absolute neutrophil count remains above at least 0.2×10⁹ cells per liter of blood during the treating of the primate.

34. A kit for treating a primate so as to enable transplantation of donor cells from a donor into the primate, the donor cells causing the production in the primate of immune system cells having at least one cellular marker that is characteristic of the donor immune system, the kit comprising:
conditioning drugs for administering a mildly myeloablative conditioning treatment to the primate;
immune blockade drugs for administering an immune blockade to the primate; and
information for using the drugs and creating a mixed chimeric immune system in the primate that is a chimer of the immune systems of the donor and the primate by a process that includes:
administering a mildly myeloablative conditioning treatment to the primate that avoids profound neutropenia in the primate, with the conditioning treatment to be started less than five days prior to the transplantation of the donor cells into the primate; and
administering an immune blockade treatment to the primate;
such that the mixed chimeric immune system is at least 0.1% donor-specific as measured in peripheral blood of the primate.

35. The kit of claim 34 wherein the information provides for a first infusion of the donor cells is infused intraportally at a concentration of at least 50×10⁶ cells/kg of recipient.

36. The kit of claim 34 wherein the information further includes instructions for administering the conditioning treatment using a total body irradiation dose of less than 500 cGy.

37. The kit of claim 34 wherein the information further includes instructions for administering the conditioning treatment using a total body irradiation dose of less than 300 cGy.

38. The kit of claim 34, wherein the conditioning drug is chosen from the group consisting of fludarabine phosphate, busulfan, and cyclophosphamide.

39. The kit of claim 34, wherein the immune blockade drug is at least one of the compounds selected from the group consisting of anti-CD40L, anti-CD40, sirolimus, CTLA4Ig, LEA29Y, and compounds that inhibit the binding of B7 to CD28.

40. A method of transplanting diabetes treating cells from a cadaveric human donor to a recipient, the method comprising:
administering diabetes treating cells from the cadaveric donor to the recipient; and
inducing a state of mixed chimerism in the recipient with a process that includes:
infusing donor cells from the donor into the recipient, the donor cells causing the production of immune system cells in the recipient having at least one cellular marker that is characteristic of the donor immune system;
administering a conditioning treatment to the recipient that is mildly myeloablative; and
administering an immune blockade treatment to the recipient.

41. The method of claim 40, wherein creating the mixed chimeric immune system is performed such that an absolute neutrophil count above at least 0.2×10⁹ cells per liter is maintained in the recipient during the step of creating the mixed chimeric immune system.

42. The method of claim 40, wherein transplanting the donor cells is performed by administering bone marrow to the recipient.

43. The method of claim 40, wherein transplanting the donor cells is performed by administering stem cells to the recipient.

44. The method of claim 40 wherein administering a mildly myeloablative conditioning treatment includes administering a total body irradiation dose of less than 500 cGy.

45. The method of claim 40 wherein administering a mildly myeloablative conditioning treatment includes administering a total body irradiation dose of less than 300 cGy.

47. The method of claim 40, further comprising pretreating the recipient with an infusion of donor antigen from the donor prior to transplantation of the donor cells.

46. The method of claim 40 wherein administering the immune blockade comprises administering sirolimus.

48. The method of claim 40, wherein the conditioning treatment includes at least one drug chosen from the group consisting of fludarabine phosphate, busulfan, and cyclophosphamide.

49. The method of claim 40, wherein administering the immune blockade treatment is accomplished by administering immune blockade agents that interfere with costimulation.

50. The method of claim 49, wherein administering immune blockade agents that interfere with costimulation is accomplished by administering at least one of the compounds selected from the group consisting of anti-CD40L, anti-CD40, sirolimus, CTLA4Ig, LEA29Y, and compounds that inhibit the binding of B7 to CD28.

51. The method of claim 40, wherein the conditioning treatment is started less than forty eight hours prior to the transplantation of the donor cells.

52. A method of transplanting diabetes treating cells from a living donor to a recipient, the method comprising:
administering diabetes treating cells from the living donor to the recipient; and
inducing a state of mixed chimerism in the recipient with a process that includes:
infusing donor cells from the living donor into the recipient, the donor cells causing the production of immune system cells in the recipient having at least one cellular marker that is characteristic of the donor immune system;
administering a conditioning treatment to the recipient that is mildly myeloablative; and
administering an immune blockade treatment to the recipient.

53. The method of claim 52 wherein the recipient receives less than 6000 islet equivalents per kg of body weight.

54. The method of claim 52 wherein the recipient receives less than 3000 islet equivalents per kg of body weight.

55. The method of claim 52 wherein the recipient receives less than 2000 islet equivalents per kg of body weight.

56. The method of claim 52, wherein a donor chimerism level of at least 5% is achieved by inducing the state of mixed chimerism as determined by measurements taken from at least one sample of the recipient's peripheral blood samples.

57. The method of claim 52, further comprising administering a cell pretreatment from the donor to the recipient prior to the infusion of the donor cells.

58. The method of claim 57, further comprising administering an antilymphocyte serum within 48 hours after an end of the cell pretreatment.

59. The method of claim 52, wherein infusing donor cells includes administering stem cells to the recipient.

60. The method of claim 59, wherein infusing donor cells includes administering stem cells to the recipient on more than one day.

61. The method of claim 52, wherein infusing donor cells includes infusing stem cells collected from a stem cell bank or the donor's peripheral blood.

62. The method of claim 52 wherein administering a mildly myeloablative conditioning treatment includes administering a total body irradiation dose of less than 500 cGy.

63. The method of claim 62 wherein administering a mildly myeloablative conditioning treatment includes administering a total body irradiation dose of less than 300 cGy.

64. The method of claim 52, wherein the conditioning treatment includes at least one drug chosen from the group consisting of fludarabine phosphate, busulfan, and cyclophosphamide.

65. The method of claim 52, wherein administering the immune blockade treatment is accomplished by administering immune blockade agents that interfere with costimulation.

66. The method of claim 65, wherein administering immune blockade agents that interfere with costimulation is accomplished by administering at least one of the compounds selected from the group consisting of anti-CD40L, anti-CD40, sirolimus, CTLA4Ig, LEA29Y, and compounds that inhibit the binding of B7 to CD28.

67. The method of claim 52, wherein inducing the state of mixed chimerism is performed such that an absolute neutrophil count in the recipient remains above at least 0.5×10⁹ cells per liter throughout the inducing of the state of mixed chimerism.

68. The method of claim 52 wherein a first infusion of the donor cells is infused intraportally at a concentration of at least 50×10⁶ cells/kg of recipient.
